(19) United States
(12) Patent Application Publication
(10) Pub. No.: US 2023/0113717 A1
(43) Pub. Date: Apr. 13, 2023

(54) Microneedle Electrodes for Biosensing

(71) Applicants: Mahla Poudineh, Waterloo (CA); Amin Ghavami Nejad, Toronto (CA); Peyman GhavamiNejad, Waterloo (CA); Sarah Odinotski, Unionville (CA)

(72) Inventors: Mahla Poudineh, Waterloo (CA); Amin Ghavami Nejad, Toronto (CA); Peyman GhavamiNejad, Waterloo (CA); Sarah Odinotski, Unionville (CA)

(21) Appl. No.: 17/963,646

(22) Filed: Oct. 11, 2022

Related U.S. Application Data
(60) Provisional application No. 63/253,739, filed on Oct. 8, 2021.

(51) Int. Cl. A61B 5/00; A61B 5/1473; A61B 5/145; A61L 31/14

(52) U.S. Cl. CPC A61B 5/685; A61B 5/1473; A61B 5/1451; A61B 5/14539; A61L 31/145; A61M 2037/0053

(57) ABSTRACT

Microneedle electrodes for detecting targets or sensing pH are described. The microneedles may be made of a hydrogel, a probe coupled to the hydrogel for generating an electrochemical signal in the presence of the target, and a conductive material for communicating the electrochemical signal through the hydrogel. The hydrogel microneedles may be used for in-situ detection of targets, such as biomolecules found in interstitial fluid. Also described are methods of producing hydrogel microneedles, articles and apparatus comprising microneedle electrodes, and methods and uses of the same. The microneedle electrodes may be used for biosensing, such as in transdermal patches for detecting biomarkers in a subject. The biosensors may be used for continuous, real-time tracking of targets in-situ, without requiring further reagents or processing steps.

### CROSS REFERENCE TO RELATED APPLICATION

This application claims priority to U.S. Provisional Patent Application No. 63/253,739 filed Oct. 8, 2021, the entire contents of which are hereby incorporated by reference.

### FIELD

The present disclosure relates generally to microneedle electrodes, their methods of production, and methods and uses thereof in biosensing.

### BACKGROUND

Transdermal biosensing can bring us one step closer to personalized and precision medicine, as it enables the continuous tracking of patient health conditions in a non- or minimally invasive manner. Transdermal biosensors analyze interstitial fluid (ISF), the fluid which is present in the lowermost skin layer of the dermis, for biomarker measurements. Compared to other body fluids, ISF has the most similar molecular composition to blood plasma, in addition to possessing other unique features including biomarkers of medical relevance. Simple and effective methods that enable the comprehensive analysis of ISF can lead to transformative advances in bio-diagnostic technologies. These approaches are not only minimally invasive and painless, but also ideally suited for point-of-care and resource-limited settings.

Microneedle (MN)-based techniques have been introduced as effective approaches for simple ISF extraction with the potential of integrating diagnostics. Different types of MNs implement various strategies to obtain ISF, for example, hollow MNs operate based on negative pressure; porous MNs use capillary force; and the most recent one, hydrogel-based MNs (HMNs) employ material absorption property. HMNs with a length less than 1000 μm and tips much sharper than hypodermic needle enable efficient piercing of the stratum corneum (outer layer of the skin) and the formation of microscale ISF extraction channels. Compared to other MNs, HMNs possess several advantages, including increased and rapid ISF extraction, high biocompatibility, lower fabrication cost, higher production yield, and ease of insertion and removal without causing skin damage.

Integrating biosensors on MNs enables in-situ ISF characterization. Hollow, metallic MN devices combined with enzymes have been implemented for real-time monitoring of various metabolites, electrolytes, and therapeutics. The main obstacles with hollow MN applications are the complex fabrication protocols and the potential risk of MN clogging. A solid hydrophobic microneedle functionalized with antibodies has been recently reported for the specific capture of target biomarkers in ISF, followed by ex vivo analysis. Although MNs functionalized with antibodies allow for on-needle biomarker detection in ISF, the sensor still needs post-processing steps, such as washing and adding detection reagents to detect targets of interest.

### SUMMARY

In one aspect there is provided a microneedle electrode comprising:

a hydrogel; a probe coupled to the hydrogel, the probe for generating an electrochemical signal in the presence of the target; and a conductive material for communicating the electrochemical signal through the hydrogel.

In one embodiment there is provided a microneedle electrode, wherein the electrochemical signal is generated in-situ.

In one embodiment there is provided a microneedle electrode, further comprising metal nanoparticles.

In one embodiment there is provided a microneedle electrode, wherein the probe comprises the metal nanoparticles.

In one embodiment there is provided a microneedle electrode, wherein the metal nanoparticles comprise platinum, silver, gold, palladium, or combinations thereof.

In one embodiment there is provided a microneedle electrode, wherein the metal nanoparticles comprise platinum and silver.

In one embodiment there is provided a microneedle electrode, wherein the probe comprises an electroactive species, a redox active species, or a combination thereof.

In one embodiment there is provided a microneedle electrode, wherein the probe comprises dopamine, conjugated dopamine, functionalized dopamine, crosslinked dopamine, metal-complexed dopamine, or combinations thereof.

In one embodiment there is provided a microneedle electrode, wherein the hydrogel comprises an optionally functionalized polymer, wherein the polymer is gelatin, hyaluronic acid, alginate, chitosan, collagen, or combinations thereof.

In one embodiment there is provided a microneedle electrode, wherein the hydrogel comprises a polymer comprising at least one C═C functionality.

In one embodiment there is provided a microneedle electrode, wherein the hydrogel comprises an acrylated polymer, a methacrylated polymer, or a combination thereof.

In one embodiment there is provided a microneedle electrode, wherein the hydrogel comprises optionally functionalized hyaluronic acid.

In one embodiment there is provided a microneedle electrode, wherein the hydrogel comprises methacrylated hyaluronic acid.

In one embodiment there is provided a microneedle electrode, wherein the hydrogel comprises dopamine-functionalized hyaluronic acid.

In one embodiment there is provided a microneedle electrode, wherein the probe coupled to the hydrogel comprises the probe being coupled to the hydrogel by a linker.

In one embodiment there is provided a microneedle electrode, wherein the probe coupled to the hydrogel comprises the probe being coupled to metal nanoparticles.

In one embodiment there is provided a microneedle electrode, wherein the probe coupled to the hydrogel comprises the probe being covalently bonded to the hydrogel.

In one embodiment there is provided a microneedle electrode, wherein the electrode is for sensing pH.

In one embodiment there is provided a microneedle electrode, wherein the electrode is for sensing the target.

In one embodiment there is provided a microneedle electrode, wherein the target is a biomolecule present in interstitial fluid.

In one embodiment there is provided a microneedle electrode, wherein the target is glucose, dopamine, uric acid, xanthine, hydrogen peroxide, potassium, or a combination thereof.

In one embodiment there is provided a microneedle electrode, wherein the target is glucose.

In one embodiment there is provided a microneedle electrode, wherein the microneedle has a length of about 300 μm to about 1000 μm, such as about 800 μm.

In one embodiment there is provided a microneedle electrode, wherein the conductive material comprises a nanoparticle, such as a metal nanoparticle, graphene, or a combination thereof.

In one embodiment there is provided a microneedle electrode, wherein the conductive material comprises a conductive polymer, such as an ionomer.

In one embodiment there is provided a microneedle electrode, wherein the conductive polymer comprises poly(3,4-ethylenedioxythiophene) polystyrene sulfonate, polyacetylene, polypyrrole, polyindole, polyaniline or combinations or copolymers thereof.

In one embodiment there is provided a microneedle electrode, wherein the conductive polymer comprises poly(3,4-ethylenedioxythiophene) polystyrene sulfonate.

In one embodiment there is provided a microneedle electrode, wherein poly(3,4-ethylenedioxythiophene) polystyrene sulfonate is present in an amount of about 30 vol % to about 35 vol %, preferably about 32 vol %, relative to the total liquid composition.

In another aspect there is provided a method of producing a microneedle, the method comprising: combining a functionalized hydrogel and a conductive material to form a mixture; adjusting the pH of the mixture; and crosslinking the mixture in a mold to form a crosslinked material.

In one embodiment there is provided a method, further comprising functionalizing a hydrogel to form the functionalized hydrogel.

In one embodiment there is provided a method, wherein adjusting the pH of the mixture and crosslinking the mixture comprises adding a base to induce crosslinking.

In one embodiment there is provided a method, wherein the base comprises a hydroxide, such as sodium hydroxide.

In one embodiment there is provided a method, wherein crosslinking the mixture comprises exposing the mixture in the mold to UV light.

In one embodiment there is provided a method, wherein crosslinking the mixture comprises one or more drying steps.

In one embodiment there is provided a method, wherein the functionalized hydrogel comprises hyaluronic acid.

In one embodiment there is provided a method, wherein functionalizing the hydrogel comprises reacting hyaluronic acid with dopamine, dopamine salts such as dopamine hydrochloride, or a combination thereof to form dopamine-functionalized hyaluronic acid.

In one embodiment there is provided a method, wherein combining the functionalized hydrogel and the conductive material comprises: dissolving about 10-100 g/L of functionalized hydrogel in water or an aqueous solution with 0.1-15 vol %, or about 0.1 vol % to about 35 vol % conductive material; and optionally, adding glycol in an amount of ^{0.5} vol %, such as about 2 vol %, or about 3 vol % (vol % relative to total liquid composition).

In one embodiment there is provided a method further comprising combining the functionalized hydrogel and the conductive material with a metal nanoparticle or metal nanoparticle precursor.

In one embodiment there is provided a method, wherein the metal nanoparticle precursor is a metal salt.

In one embodiment there is provided a method, wherein the metal salt is silver nitrate and/or platinum sodium chloride.

In one embodiment there is provided a method, wherein the metal nanoparticle or metal nanoparticle precursor is added in an amount of 1-5 wt %.

In one embodiment there is provided a method, further comprising degassing the mixture in the mold.

In another aspect there is provided a microneedle electrode obtainable or obtained by the method according to an embodiment herein disclosed.

In another aspect there is provided an apparatus for biosensing, the apparatus comprising: the microneedle electrode according to an embodiment herein disclosed; a reference electrode; a counter electrode; and a detector for detecting the electrochemical signal.

In one embodiment there is provided an apparatus, wherein the detector is a potentiostat.

In another aspect there is provided a transdermal patch comprising the microneedle electrode according to an embodiment herein disclosed.

In one embodiment there is provided a transdermal patch, wherein the microneedle electrode is a working electrode, and the transdermal patch further comprises a reference electrode and a counter electrode.

In one embodiment there is provided a transdermal patch, wherein the reference electrode is a microneedle electrode.

In one embodiment there is provided a transdermalpatch, wherein the reference electrode comprises Ag/AgCl.

In one embodiment there is provided a transdermal patch, wherein the counter electrode is a microneedle electrode.

In one embodiment there is provided a transdermal patch, wherein the counter electrode comprises Au.

In another aspect there is provided a method for transdermal biosensing of a target in a subject, the method comprising: applying the transdermal patch according to an embodiment herein disclosed to the skin of the subject; detecting the electrochemical signal; and associating the electrochemical signal to pH or to the concentration of the target in the subject.

In one embodiment there is provided a method, wherein detecting the electrochemical signal is in-situ.

In one embodiment there is provided a method, wherein detecting the electrochemical signal is reagentless.

In one embodiment there is provided a method, wherein detecting the electrochemical signal occurs without requiring removal of the transdermal patch.

In one embodiment there is provided a method, wherein detecting the electrochemical signal occurs while the transdermal patch is applied to the subject.

In one embodiment there is provided a method, wherein detecting the measurable signal occurs in the absence of further processing of the transdermal patch.

In one embodiment there is provided a method, wherein detecting the electrochemical signal comprises measuring voltammetry or amperometry.

In one embodiment there is provided a method, wherein associating the electrochemical signal comprises comparing a characteristic of the electrochemical signal to a calibration curve of measured electrochemical signals of known pH or of known concentration of the target.

### BRIEF DESCRIPTION OF THE FIGURES

Embodiments of the present disclosure will now be described, by way of example only, with reference to the attached Figures.

FIG. 1 depicts a microneedle according to an embodiment of the present disclosure.

FIG. 2 depicts a schematic representation of a glucose sensing strategy and HMN-pH and glucose meter fabrication.

FIG. 3 shows some characterization data for a dopamine-functionalized hyaluronic acid, poly(3,4-ethylenedioxythiophene) polystyrene sulfonate (DAHA PEDOT:PSS) Pt hydrogel.

FIG. 4 depicts in vitro characterization of a hydrogel microneedle-based glucose biosensor.

FIG. 5 depicts an ¹H NMR of DAHA which shows peals at 6.95-6.60 ppm which confirms the aromatic rings of DA.

FIG. 6 depicts absorption spectra of DAHA in a polymer product with different conjugation rates (10%, 17%, and 25%).

FIG. 7 depicts XPS measurements of exemplary compositions, namely, DAHA, DAHA PEDOT:PSS, DAHA PEDOT:PSS Ag, DAHA PEDOT Pt and DAHA PEDOT Ag Pt.

FIG. 8 depicts a SEM image of a microneedle array with 25% DAHA.

FIG. 9 is a schematic representation of an in-situ pH sensing strategy and HMN-pH meter fabrication.

FIG. 10 shows characterization and optimization of a HMN polymer and needles.

FIG. 11 depicts in vitro and ex vivo characterization of pH sensing for a three-electrode system.

FIG. 12 depicts a device according to an embodiment of the present disclosure, for real-time, continuous tracking of a target.

FIG. 13 depicts electrical and material characterization of the HMN polymer and needles.

FIG. 14 depicts SEM and optical characterization of the HMN polymer and needles.

FIG. 15 depicts an in-vivo pH sensing model using the three MN electrode system.

FIG. 16 depicts characterization of the formation of Pt NPs within the composite hydrogel.

FIG. 17 depicts in-vivo glucose detection in diabetic rats.

FIG. 18 depicts image analysis pipeline used in lmageJ for porosity measurements of the (i) 0%, (ii) 2.5%, and (iii) 5% PEDOT:PSS HMN polymer network. A) Raw SEM images. B) 8-bit mask conversions. C) Regions analyzed for pore area measurement. Scale for all images is 40 μm.

FIG. 19 depicts 3D SEM images of a) 10% and b) 15% PEDOT:PSS HMNs showing poor needle integrity as a result of high PEDOT:PSS concentrations.

FIG. 20 depicts H&E staining showing HMN penetration sites and surrounding subcutaneous tissue. a) 0 mins after HMN removal b) 10 mins after HMN removal c) 30 mins after HMN removal d) control with no HMN application

FIG. 21 depicts sample in-vivo amperometric responses of the HMN three MN electrode system at 0.4 V.

FIG. 22 depicts linear portion of the ex-vivo calibration used for HMN-WE validation. Data reported as mean±SEM with n=4 replicates. R2=0.6882 as analyzed by simple linear regression.

FIG. 23 depicts EDC mapping analysis DHP Ag-Pt composite hydrogel shows the formation of nanoparticles.

FIG. 24 depicts In-vivo glucose detection in diabetic rats.

FIG. 25: depicts in-vitro and ex-vivo characterization of pH sensing using the 5% PEDOT:PSS HMN three-electrode system.

FIG. 26 depicts rigidity of HMN electrodes a) before insertion b) after 1st insertion c) after 2nd insertion d) after 3rd insertion a) after 4th insertion.

FIG. 27 depicts in-vitro amperometric response of the three MN electrode system on pH-controlled agarose hydrogels at 0.4 V. Data presented as the mean (n=1).

FIG. 28 depicts a comparison of the HMN signal stability after multiple-day storage in porcine skin. Data expressed as mean±SEM with n=3 replicates and normalized to day 0 measurement. *P=0.0154, ns (not significant), as analyzed by ordinary one-way ANOVA with multiple comparisons follow up t-tests using Tukey's correction.

FIG. 29 depicts a comparison of the HMN signal reproducibility after multiple insertions into porcine skin. Data expressed as mean±SEM with n=3 replicates and normalized to insertion 1.

FIG. 30 depicts Ex-vivo glucose detection using HMN-CGM device.

FIG. 31 depicts cyclic voltammetry (CV) measurement in agarose hydrogel. HMN-CGM device was applied into agarose hydrogel loaded with different concentrations of glucose and the capability of sensor for glucose measurement was studied using CV scanning. The bar graph shows the glucose oxidation peaks at different concentrations.

FIG. 32 depicts cyclic voltammetry (CV) measurement in porcine skin. HMN-CGM device was applied into porcine skin equilibrated with different concentrations of glucose and the capability of sensor for glucose measurement was studied using CV scanning. The bar graph shows the glucose oxidation peaks at different concentrations.

FIG. 33 depicts EIS Measurement of HMN-WE inserted into the porcine skin for different durations (left). Fitted values of charge transfer resistance±SD (n=4) (right).

FIG. 34 depicts Left, porcine skin under bending (i) or twisting (ii) deformation. Right, the electrochemical signal measurement under the cyclic deformation. Inset shows a zoom-in region of the graph between 35 to 55 s. The chronoamperometry measurements were performed at 0.4 V for 100 s. The experiments were carried out upon 100 min-insertion of the HMN-WE in the skin to take account for the time required for the HMN patch to reach its maximum swelling.

### DETAILED DESCRIPTION

Analyzing interstitial fluid (ISF) via microneedle (MN) devices can enable patient health monitoring in a minimally invasive manner and at point-of-care (POC) settings. However, most MN-based diagnostic approaches require complicated fabrication processes or post-processing of the extracted ISF. Described herein is an in-situ and on-needle measurement of target analytes performed by integrating hydrogel microneedles (HMN) with a conductive material and an electrochemical probe. In one example, dopamine-functionalized hyaluronic acid (DA-HA) is used in combination with a conductive polymer poly(3,4-ethylenedioxythiophene) polystyrene sulfonate (PEDOT:PSS) in a hydrogel network to enable boosting the electrical properties of MN, making the patch suitable for the working electrode of the biosensor. In another example, DA-HA and PEDOT:PSS are used in combination with metal nanoparticles, such as platinum and/or silver nanoparticles, for non-enzymatically sensing of glucose in ISF. It is expected that the microneedle electrodes herein disclosed may be used for continuous, real-time, biosensing of pH or other targets.

In one or more embodiments, the present disclosure provides continuous and real-time biosensors that can provide insight into the health status of subjects and their response to therapeutics, in a non- or minimally invasive manner. For example, continuous glucose meters (CGMs) have boosted diabetes care by emancipating millions of diabetic patients from the need for repeated self-testing by pricking their fingers every few hours. However, CGMs still suffer from deficiencies with regard to accuracy, precision, and stability. This is mainly due to their dependency on an enzymatic detection mechanism. Current CGMs rely on the glucose oxidase enzyme to catalyze the oxidation of β-D-glucose to produce gluconic acid and hydrogen peroxide, which are detected electrochemically. Described herein is a dopamine conjugated hyaluronic acid-based microneedle(DA-HA-MN)-based biosensor for in situ, continuous, real-time, and enzyme-free sensing of a target such as biomolecules (e.g., glucose) and/or for pH sensing, The MN-based biosensor according to one or more embodiments may be made by DA-HA hydrogel which can extract dermal Interstitial Fluid (ISF) effectively upon insertion to skin. The DA-HA-MN biosensors according to one or more embodiments herein disclosed can be used to fabricate a MN patch composed of DA conjugated with HA hydrogel network which can be crosslinked by adjusting the patch' pH without the need for UV irridation or addition of reagents.

In one or more embodiments, platinum (Pt) nanoparticles and/or silver (Ag) nanoparticles (NPs) were also synthesized within three dimensional (3D) porous hydrogel scaffolds for non-enzymatically sensing of the glucose in ISF. The incorporation of a water dispersible conductive polymer, such as poly(3,4-ethylenedioxythiophene) polystyrene sultanate (PEDOT:PSS), into the hydrogel network was found to improve the electrical properties of MN, making the patch according to one or more embodiments suitable for use as a working electrode of a biosensor. For glucose sensing, an exemplary DAHA-PEDOT:PSS-Pt MNs may be activated by miniaturized Potentiostat where it may be used as the working electrode (WE) along with, for example, Gold MN array and Silver/SilverChloride MN Array for Counter and Reference electrode (CE,RE), respectively. Without embedding the NPs, such a biosensor can also act as a pH sensor due to dopamine's cathecol moiety electron transfer property upon being in different pH buffers. The continuous glucose and pH measurement by this biosensor may occur without being affected by other common interferents. In one example of using the MN-based three-electrode system and a miniaturized Potentiostat it is possible to sense different glucose concentrations and changes in pH in vitro.

The need for real-time, enzyme-less and continuous monitoring of biomarkers, such as glucose, is tightly linked with various challenges and difficulties. Because of the continuous and real-time measurement, the biosensor should remain stable even after prolonged exposure to complex biological environments. The sensing mechanism should have adequate sensitivity, specificity and dynamic range in a short time-scale. The biosensor should not utilize any enzyme for sensing. The biosensor should be compatible with wearable and point-of-care (POC) devices. Finally, the device is preferably fabricated using an easy process with no need for clean room facilities.

CGMs utilizing glucose oxidase enzyme are prone to false measurement upon exposure to different environment conditions, such as change in temperature, humidity, pH, etc. Other electrochemical biosensors can continuously measure glucose and pH in vivo, including a platform for continuous detection of glucose in the ISF of live animals. However, the approach of coating or attaching nanoparticles on a rigid MN electrode array only enables the sensing on the surface of the MN-based platform, which has a limited sensitivity. In such a system, the rigid and non-porous microneedles are unable to quickly absorb ISF toward their sensory surfaces for a fast and sensitive measurement.

Microneedle-based transdermal devices are emerging to address the challenge of non/minimally invasive wearable biosensing, and can be potentially employed for POC diagnosis and tracking, Microneedles (MN) enable ready access to dermal interstitial fluid (ISF), one of the more prevalent, accessible fluids in the body that contains important biomarkers for continuous monitoring. Advances have recently been made in exploiting hollow, metallic MN-based devices for real-time monitoring of various metabolites, electrolytes, and therapeutics and toward the simultaneous multiplexed detection of key chemical markers. However, these sensors tend to be mainly limited to enzymatic based detection, which can hinder their performance for detection of analytes for which enzymes are not available. It has been found that hydrogel microneedles (HMNs), which have been mainly used for cosmetics and drug delivery applications, have potential for diagnostics where extracted ISF has been used for off-chip detection of different analytes. Indeed, HMN arrays are considered to possess several advantages, such as increased amount of ISF (10 μL vs 2 μL in hollow MN), lower fabrication cost and higher production yield when comparing to other MNs. However, they lacked in-situ sensing.

In one or more embodiments of the present disclosure, the herein described conductive HMN arrays may address the foretold limitation of the real-time biosensing. The present disclosure may address the above-noted challenges for at least the following reasons. In one example, Pt and/or Ag NPs enable real-time, continuous and enzyme-less measurement of glucose. The HMN biosensors can be also selected for high sensitivity and specificity in target detection because of their 3D conductive hydrogel grid. Incorporating an integrated chip (IC) and supporting electronics for miniaturized electrochemical measurement and wireless transmission of data can enable monitoring in POC setting. And finally, using microneedle electrodes as herein described, such as a DA-HA hydrogel, simplifies the fabrication process.

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein refers to the list that follows being non-exhaustive and may or may not include any other additional suitable items, for example one or more further feature(s), component(s) and/or ingredient(s) as appropriate.

The term “subject”, as used herein, refers to an animal, and can include, for example, domesticated animals, such as cats, dogs, etc., livestock (e.g., cattle, horses, pigs, sheep, goats, etc.), laboratory animals (e.g., mouse, rabbit, rat, guinea pig, etc.), mammals, non-human mammals, primates, non-human primates, rodents, birds, reptiles, amphibians, fish, and any other animal. In a specific example, the subject is a human.

Generally, the present disclosure provides microneedle electrodes for detecting a target. In another aspect, the present disclosure provides methods of producing microneedles. In another aspect, the present disclosure provides methods and apparatus using the microneedles herein disclosed. In other aspects, the present disclosure provides transdermal patches and methods of biosensing using the same.

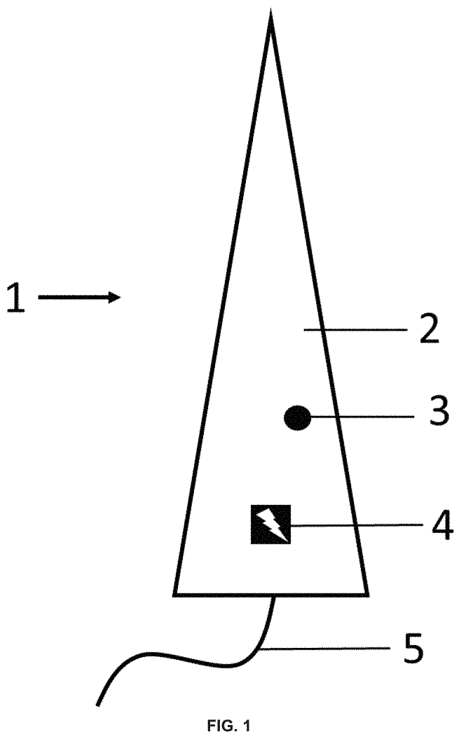

According to one or more embodiments, the microneedle electrode of the present disclosure may comprise a hydrogel, a probe coupled to the hydrogel, the probe for generating an electrochemical signal in the presence of the target, and a conductive material for communicating the electrochemical signal through the hydrogel. The electrochemical signal may be generated in-situ. In other words, the signal may be measurable without requiring other reagents (e.g. reagent-less) or processing steps. FIG. 1 shows an embodiment of a microneedle electrode (1) comprising a hydrogel (2) and a probe (3). In FIG. 1, the conductive material (4) communicates the electrochemical signal of the probe to a wire (5).

### Hydrogels

Microneedle electrodes described herein comprise a hydrogel. The hydrogel may be any suitable polymer or combinations thereof. The hydrogel preferably comprises functionalities that allow for crosslinking and/or functionalization, and it will be understood that any polymer with such functionalities may be used. The hydrogel may comprise an optionally functionalized polymer. Suitable optionally functionalized polymers include but are not limited to gelatin, hyaluronic acid, alginate, chitosan, collagen, and combinations thereof. The hydrogel may include a combination of polymers, and said polymers may have differing functionalities. The hydrogel may comprise functionalized hyaluronic acid, such as dopamine-functionalized hyaluronic acid. The hydrogel may comprise a polymer comprising at least one C═C functionality, where the C═C functionality may allow for crosslinking and/or functionalization. The hydrogel may comprise an acrylated polymer, a methacrylated polymer, or a combination thereof. The hydrogel may comprise methacrylated hyaluronic acid. The hydrogel may comprise any suitable functionality for generating an electrochemical signal, such as an electroactive species or a redox active moiety.

### Probes

Microneedles described herein comprise a probe. The probe may be for generating a measurable signal, such as an electrochemical signal, in the presence of a target, alone or in communication with other components of the microneedle. The probe may be coupled to the hydrogel by any suitable means or fashion, such as by bond, association, or complexation. The probe may be coupled to the hydrogel directly, or by a linker. If the probe is coupled to the hydrogel by a linker, the linker may be formed from any suitable functional group. The probe may be linked to a nanoparticle. The probe may comprise a metal nanoparticle. The probe may be covalently bonded to the hydrogel. The probe may be a functional group of the functionalized hydrogel.

Any suitable probe may be used in the microneedle electrodes herein described. The probe may be any suitable moiety for generating a measurable signal. The probe preferably generates a measurable signal when bound to, associated with, or in proximity to the target. The probe may be an electroactive species, a redox active species, or a combination thereof. The probe may comprise a catechol moiety. The probe may be dopamine or a derivative of dopamine. The probe may be dopamine, conjugated dopamine, functionalized dopamine, crosslinked dopamine, metal-complexed dopamine, or combinations thereof. The probe may comprise metal nanoparticles. In one or more embodiments, the metal nanoparticles may comprise platinum, silver, gold, palladium, or combinations thereof. The metal nanoparticles may comprise platinum and/or silver.

### Other Components

The microneedles herein described may comprise other components. For example, the microneedle or the hydrogel may comprise a nanoparticle, such as a metal nanoparticle. In one or more embodiments, the probe may be coupled to the nanoparticle, and the nanoparticle coupled or associated with the hydrogel. In one or more embodiments, the nanoparticle may be the conductive material or a component thereof. In one or more embodiments, the nanoparticle may communicate, or assist with communicating, the electrochemical signal through the hydrogel. In one or more embodiments, the nanoparticle may enhance the electrical properties of the microneedle. The nanoparticle may comprise any suitable metal, such as platinum, silver, gold, palladium, or combinations thereof. The metal nanoparticles may be platinum nanoparticles or platinum and silver nanoparticles. Graphene may be used in addition to, or in place of, metal nanoparticles in the microneedle. The microneedle may comprise any other suitable components.

### Measurable Signal

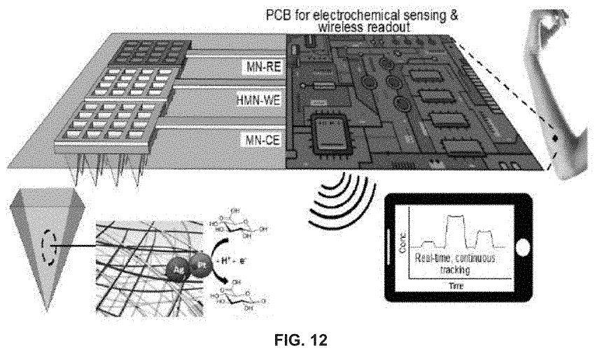

The microneedles herein described are for generating a measurable signal in the presence of a target, such as a biomolecule or a biomarker. The measurable signal may be an electrochemical signal. The electrochemical signal may be generated in-situ by the probe. The electrochemical signal may be communicated through the hydrogel, such as with the assistance of a conductive material. It will be understood that other measurable signals may be used without departing from the spirit of the present disclosure. The measurable signal may be detectable by a platform for continuous, real-time measurement, such as the platform depicted in FIG. 12.

### Targets

The microneedles and transdermal patches herein described may be used to detect one or more targets. The target may be any suitable molecule or ion for detection. The target may be a biomolecule or biomarker, such as a biomolecule present in interstitial fluid. The electrode may be for sensing pH, in which case the target may be considered a proton. The electrode may be for sensing a target, such as glucose, dopamine, uric acid, xanthine, hydrogen peroxide, or potassium. In one embodiment, the target is glucose. It will be understood that any molecule or ion suitable for generating an electrochemical signal in the presence of the probe may be detected, such as a molecule or ion that undergoes a redox reaction with the probe.

### Conductive Materials

The microneedles herein described comprise a conductive material. The conductive material may be any suitable material for communicating a signal from the probe through the hydrogel. For example, the conductive material may communicate an electrochemical signal of the probe through the hydrogel and to an electrical wire associated with the microneedle, The conductive material may be a nanoparticle, such as a metal nanoparticle, or graphene. The nanoparticle or graphene may be embedded within the hydrogel's 3D network. The conductive material may be a conductive polymer, such as an ionomer. Any suitable conductive polymer may be used, such as poly(3,4-ethylenedioxythiophene) polystyrene sulfonate, polyacetylene, polypyrrole, polyindole and polyaniline and their copolymers. In one or more embodiments the conductive polymer comprises poly(3,4-ethylenedioxythiophene) polystyrene sulfonate, such as in an amount of about 30 vol % to about 35 vol %, preferably about 32 vol %, relative to the total liquid composition. The conductive material or polymer may be mixed with, embedded in, or directly linked (e.g. by crosslinking) to the hydrogel.

### Methods of Producing Microneedles

In one or more aspects, there is provided a method of producing a microneedle. The method may comprise combining a functionalized hydrogel and a conductive material to form a mixture and forming said mixture into the microneedle. The method may comprise curing the mixture, such as by crosslinking the mixture in a mold to form a crosslinked material. The crosslinking may be induced by any suitable means, such as by adjusting the pH or exposing the mixture to UV light. In one embodiment, the method comprises combining a functionalized hydrogel and a conductive material to form a mixture; adjusting the pH of the mixture; and crosslinking the mixture in a mold to form a crosslinked material. The method may further comprise functionalizing a hydrogel to form the functionalized hydrogel, such as by functionalizing hyaluronic acid with dopamine, dopamine salts such as dopamine hydrochloride, or a combination thereof to form dopamine-functionalized hyaluronic acid. Adjusting the pH may occur at any suitable step or stage in the method, and may involve acidifying or basifying at least one component. Adjusting the pH may include adding a base to one or more of the hydrogel, the conductive material, or a combination thereof, to induce crosslinking. The base may be any suitable base, such as a hydroxide, for example sodium hydroxide. Crosslinking may be done in the absence of light, or may involve exposing the mixture to UV light. The method may comprise one or more drying steps, such as drying the mixture in the mold before or after crosslinking. The functionalized hydrogel may be functionalized hyaluronic acid, such as dopamine-functionalized hyaluronic acid. Combining the functionalized hydrogel and the conductive material may be done by any suitable means or procedure. For example, the method may include dissolving about 10 g/L to about 100 g/L, such as about 50 g/L or about 83 g/L (50 mg/patch), of functionalized hydrogel in water or an aqueous solution with 0.1-15 vol %, or about 0.1 vol % to about 35 vol % conductive material; and optionally, adding glycol in an amount of 0-5 vol %, such as about 2 vol %, or about 3 vol % (vol % relative to total liquid composition). The method may further include degassing, such as degassing the mixture in the mold by any suitable means, such as by centrifuge, sonication, and or applying vacuum. The method may further comprise combining the functionalized hydrogel and the conductive material with a metal nanoparticle or metal nanoparticle precursor. The metal nanoparticle precursor may be a metal salt, such as a Pt, Pd, Au, or Ag salt. The metal nanoparticle may be a metal nitrate or metal chloride, for example, silver nitrate and/or platinum sodium chloride. The metal nanoparticle precursor may be added in any suitable amount such as about 1-5 wt %.

### Properties of Microneedles

In one or more aspects, there is provided a microneedle obtainable or obtained by the methods herein disclosed. The microneedle may have a length of about 300 μm to about 1000 μm, such as about 800 μm. The microneedle may be produced as a plurality of microneedles. The plurality of microneedles may be in any suitable arrangement, such as a grid. The plurality of microneedles may be connected, for example, the microneedles may be cast by a mold that includes a base layer from which the microneedles extend. The base layer may comprise other components, such as an electrical wire or connection. The plurality of microneedles may be in a grid of about 10 to about 500. The plurality of microneedles may be in a square grid, such as a grid of 3×3, or 4'4, or 5×5, etc. The plurality of microneedles may be in a rectangular grid, such as a grid of 3×4, or 3×5, or 3'6, etc. or 4×5, or 4×6, etc. The plurality of microneedles may be in an irregularly shaped grid, or any suitable shape or pattern. The plurality of microneedles may be individually spaced apart, such as at a distance of about 100 μm to about 1000 μm, such as about 500 μm.

### Apparatus

In one or more aspects, there is provided an apparatus for detecting a target in a sample. The apparatus may comprise a microneedle as disclosed herein together with a detector for detecting the electrochemical signal. The detector may be any suitable detector, such as a potentiostat. The apparatus may further comprise a reference electrode and/or a counter electrode, or they may be provided separately. The sample may be any suitable sample, such as a solution comprising the target, a biological sample or solution thereof. The sample may have been taken from a subject, or the apparatus may be used with a transdermal patch for biosensing, wherein the sample is the subject's interstitial fluid.

### Transdermal Patch

In one or more aspects, there is provided a transdermal patch comprising a microneedle according to one or more embodiments of the present disclosure. In the transdermal patch, the microneedle electrode may be the working electrode. The transdermal patch may further comprise a reference electrode and/or a counter electrode, or these electrodes may be provided separately, such as on a separate patch. The reference electrode and/or the counter electrode may also be microneedle electrodes, or they may be any suitable electrode for use in the system. The reference electrode may be a microneedle electrode comprising Ag/AgCl. The counter electrode may be a microneedle electrode comprising Au. The transdermal patch may comprise three electrodes (a working electrode, counter electrode, and reference electrode) or a plurality of each. The transdermal patch may comprise other components, such as adhesive to attach the patch and/or a bandage. The electrodes may be localized on one area of the patch, such as in a side-by-side arrangement. Each electrode may have its own associated wiring, The arrangement of the electrodes may be, for example, as demonstrated in FIG. 12, or any suitable arrangement.

### Biosensing

In one or more aspects there is provided a method for transdermal biosensing, or a use of the microneedles herein disclosed for transdermal biosensing. The method may comprise applying a transdermal patch or any suitable means of applying microneedles as herein described. The method may comprise applying the transdermal patch to any suitable location of a subject, such as arm, leg, abdomen, etc. The method or use for biosensing may include detecting the measurable signal (electrochemical signal) and associating the measurable signal to the concentration of the target in the subject. Alternatively, the method or use may be for simply identifying the presence of a given level of the target in the subject, as opposed to the exact concentration. Detecting the measurable signal may be done completely in-situ; in other words, the detection may be done without further processing steps. Detecting may be reagentless. Detecting may occur without requiring removal of the transdermal patch, or without further processing of the transdermal patch. Detecting may occur while the transdermal patch is applied, or in contact with the subject. Detecting may occur in the absence of further processing of the transdermal patch. Detecting the electrochemical signal may comprise measuring voltammetry or amperometry, or any other suitable measurement of the microneedle system. Associating the electrochemical signal may comprise comparing a measured intensity of the signal to a calibration curve of measured intensities of known concentrations of the target or of known pH. It will be understood that any suitable means of associating the measurable signal to the presence or absence (or concentration) of the target may be used.

Herein, there is described:

1. A microneedle electrode comprising:
a hydrogel;
a probe coupled to the hydrogel, the probe for generating an electrochemical signal in the presence of the target; and
a conductive material for communicating the electrochemical signal through the hydrogel.
   2. The microneedle electrode of item 1, wherein the electrochemical signal is generated in-situ.
   3. The microneedle electrode of item 1 or 2, further comprising metal nanoparticles.
   4. The microneedle electrode of item 3, wherein the probe comprises the metal nanoparticles.
   5. The microneedle electrode of item 3 or 4, wherein the metal nanoparticles comprise platinum, silver, gold, palladium, or combinations thereof.
   6. The microneedle electrode of item 4, wherein the metal nanoparticles comprise platinum and silver.
   7 The microneedle electrode of any one of items 1 to 6, wherein the probe comprises an electroactive species, a redox active species, or a combination thereof.
   8. The microneedle electrode of any one of items 1 to 7, wherein the probe comprises dopamine, conjugated dopamine, functionalized dopamine, crosslinked dopamine, metal-complexed dopamine, or combinations thereof.
   9. The microneedle electrode of any one of items 1 to 8, wherein the hydrogel comprises an optionally functionalized polymer, wherein the polymer is gelatin, hyaluronic acid, alginate, chitosan, collagen, or combinations thereof.
   10. The microneedle electrode of any one of items 1 to 9, wherein the hydrogel comprises a polymer comprising at least one C═C functionality.
   11. The microneedle electrode of any one of items 1 to 10, wherein the hydrogel comprises an acrylated polymer, a methacrylated polymer, or a combination thereof.
   12. The microneedle electrode of any one of items 1 to 11, wherein the hydrogel comprises optionally functionalized hyaluronic acid.
   13. The microneedle electrode of any one of items 1 to 12, wherein the hydrogel comprises methacrylated hyaluronic acid.
   14. The microneedle electrode of any one of items 1 to 13, wherein the hydrogel comprises dopamine-functionalized hyaluronic acid.
   15. The microneedle electrode of any one of items 1 to 14, wherein the probe coupled to the hydrogel comprises the probe being coupled to the hydrogel by a linker.
   16. The microneedle electrode of any one of items 1 to 15, wherein the probe coupled to the hydrogel comprises the probe being coupled to metal nanoparticles.
   17. The microneedle electrode of any one of items 1 to 16, wherein the probe coupled to the hydrogel comprises the probe being covalently bonded to the hydrogel.
   18. The microneedle electrode of any one of items 1 to 17, wherein the electrode is for sensing pH.
   19. The microneedle electrode of any one of items 1 to 18, wherein the electrode is for sensing the target.
   20. The microneedle electrode of any one of items 1 to 19, wherein the target is a biomolecule present in interstitial fluid.
   21. The microneedle electrode of any one of items 1 to 20, wherein the target is glucose, dopamine, uric acid, xanthine, hydrogen peroxide, potassium, or a combination thereof.
   22. The microneedle electrode of any one of items 1 to 21, wherein the target is glucose.
   23. The microneedle electrode of any one of items 1 to 22, wherein the microneedle has a length of about 300 μm to about 1000 μm, such as about 800 μm.
   24. The microneedle electrode of any one of items 1 to 23, wherein the conductive material comprises a nanoparticle, such as a metal nanoparticle, graphene, or a combination thereof.
   25 The microneedle electrode of any one of items 1 to 24, wherein the conductive material comprises a conductive polymer, such as an ionomer.
   26. The microneedle electrode of item 25, wherein the conductive polymer comprises poly(3,4-ethylenedioxythiophene) polystyrene sulfonate, polyacetylene, polypyrrole, polyindole, polyaniline or combinations or copolymers thereof.
   27. The microneedle electrode of item 25 or 26, wherein the conductive polymer comprises poly(3,4-ethylenedioxythiophene) polystyrene sulfonate.
   28. The microneedle electrode of any one of items 25 to 27, wherein poly(3,4-ethylenedioxythiophene) polystyrene sulfonate is present in an amount of about 30 vol % to about 35 vol %, preferably about 32 vol %, relative to the total liquid composition.
   29. A method of producing a microneedle, the method comprising:
combining a functionalized hydrogel and a conductive material to form a mixture;
adjusting the pH of the mixture; and
crosslinking the mixture in a mold to form a crosslinked material.
   30. The method of item 29, further comprising functionalizing a hydrogel to form the functionalized hydrogel.
   31. The method of item 29 or 30, wherein adjusting the pH of the mixture and crosslinking the mixture comprises adding a base to induce crosslinking.
   32. The method of item 31, wherein the base comprises a hydroxide, such as sodium hydroxide.
   33. The method of any one of items 29 to 32, wherein crosslinking the mixture comprises exposing the mixture in the mold to UV light.
   34. The method of any one of items 29 to 33, wherein crosslinking the mixture comprises one or more drying steps.
   35. The method of any one of items 29 to 34, wherein the functionalized hydrogel comprises hyaluronic acid.
   36. The method of item 30, wherein functionalizing the hydrogel comprises reacting hyaluronic acid with dopamine, dopamine salts such as dopamine hydrochloride, or a combination thereof to form dopamine-functionalized hyaluronic acid.
   37. The method of any one of items 29 to 36, wherein combining the functionalized hydrogel and the conductive material comprises:
dissolving about 10-100 g/L of functionalized hydrogel in water or an aqueous solution with 0.1-15 vol %, or about 0.1 vol % to about 35 vol % conductive material; and
optionally, adding glycol in an amount of 0-5 vol %, such as about 2 vol % or about 3 vol % (vol % relative to total liquid composition).
   38. The method of any one of items 29 to 37, further comprising combining the functionalized hydrogel and the conductive material with a metal nanoparticle or metal nanoparticle precursor.
   39. The method of item 38, wherein the metal nanoparticle precursor is a metal salt.
   40. The method of item 39, wherein the metal salt is silver nitrate and/or platinum sodium chloride.
   41. The method of any one of items 38 to 40, wherein the metal nanoparticle or metal nanoparticle precursor is added in an amount of 1-5 wt %.
   42. The method of any one of items 29 to 41, further comprising degassing the mixture in the mold.
   43. A microneedle electrode obtainable or obtained by the method according to any one of items 29 to 42.
   44. An apparatus for biosensing, the apparatus comprising:
the microneedle electrode according to any one of items 1 to 28 or 43;
a reference electrode;
a counter electrode; and
a detector for detecting the electrochemical signal.
   45. The apparatus of item 44, wherein the detector is a potentiostat.
   46. A transdermal patch comprising the microneedle electrode according to any one of items 1 to 28 or 43.
   47. The transdermal patch of item 46, wherein the microneedle electrode is a working electrode, and the transdermal patch further comprises a reference electrode and a counter electrode.
   48. The transdermal patch of item 47, wherein the reference electrode is a microneedle electrode.
   49. The transdermal patch of item 48, wherein the reference electrode comprises Ag/AgCl.
   50. The transdermal patch of any one of items 47 to 49, wherein the counter electrode is a microneedle electrode.
   51. The transdermal patch of item 50, wherein the counter electrode comprises Au. 52. A method for transdermal biosensing of a target in a subject, the method comprising:
applying the transdermal patch according to any one of items 46 to 51 to the skin of the subject;
detecting the electrochemical signal; and
associating the electrochemical signal to pH or to the concentration of the target in the subject.
   53. The method of item 52, wherein detecting the electrochemical signal is in-situ.
   54. The method of item 52 or 53, wherein detecting the electrochemical signal is reagentless.
   55. The method of any one of items 52 to 54, wherein detecting the electrochemical signal occurs without requiring removal of the transderrnal patch.
   56. The method of any one of items 52 to 55, wherein detecting the electrochemical signal occurs while the transdermal patch is applied to the subject.
   57. The method of any one of items 52 to 56, wherein detecting the measurable signal occurs in the absence of further processing of the transdermal patch.
   58. The method of any one of items 52 to 57, wherein detecting the electrochemical signal comprises measuring voltammetry or amperometry.
   59. The method of any one of items 52 to 58, wherein associating the electrochemical signal comprises comparing a characteristic of the electrochemical signal to a calibration curve of measured electrochemical signals of known pH or of known concentration of the target.

To gain a better understanding of the invention described herein, the following examples are set forth. It should be understood that these examples are for illustrative purposes only. Therefore, they should not limit the scope of this invention in anyway.

### EXAMPLES

### Example 1: A Hydrogel Microneedle-Based Biosensor for Continuous, Real-Time, and Enzyme-Less Glucose Measurement

### 1.1 Abstract

Continuous glucose meters (CGMs) have boosted diabetes care by emancipating millions of diabetic patients the need for repeated self-testing by pricking their fingers every few hours. However, CGMs still suffer from major deficiencies with regard to accuracy, precision, and stability. This is mainly due to their dependency on an enzymatic detection mechanism. Current CGMs rely on the glucose oxidase enzyme to catalyze the oxidation of 3-D-glucose to produce gluconic acid and hydrogen peroxide which are detected electrochemically. Herein, the first Microneedle(MN)-based glucose and pH biosensor is introduced, using swelling hydrogel for in situ, continuous, real-time, and enzyme-free sensing. The MN-based biosensor is made by swellable crosslinked dopamine (DA)-hyaluronic acid (HA) hydrogel which can extract dermal Interstitial Fluid (ISF) effectively upon insertion to the skin. Platinum (Pt) nanoparticles (NPs) have been synthesized within three-dimensional (3D) porous hydrogel scaffolds for non-enzymatically sensing of the glucose in ISF. The incorporation of a highly water dispersible conductive polymer, poly(3,4-ethylenedioxythiophene) polystyrene sulfonate (PEDOT:PSS) into the hydrogel network has enabled boosting the electrical properties of MN, making the patch suitable for the working electrode of the biosensor, For glucose sensing, the DAHA-PEDOT:PSS-Pt MNs will be activated by miniaturized Potentiostat where it acts as the working electrode (WE) along with Gold MN array and Silver/SilverChloride MN Array for Counter and Reference electrode (CE, RE), respectively. Without embedding the NPs, this biosensor can also act as a pH sensor due to the dopamine's catechol moiety electron transfer property upon being in different pH buffers. The continuous glucose measurement by this biosensor will not be affected by other common interferents. Using the MN-based three-electrode system and a miniaturized Potentiostat, it is possible to sense different glucose concentrations in vitro.

### 1.2 Introduction

Diabetes mellitus is a metabolic disease that causes the body to fail to control the blood glucose level. The fluctuation in blood glucose levels of people with type 1 and type 2 diabetes can gradually deteriorate cardiovascular, neuropsychological, and miscellaneous system function. The routine method for controlling this disease is to periodically check the blood glucose level and regulate it by injecting insulin, taking oral drugs, or balancing dietary and physical habits. Currently, the most common method in order to monitor the glucose level is finger-prick glucose monitoring. This method can accurately measure the blood glucose level requiring a small amount of blood. However, the finger-prick method is unable to show the rate and direction of glucose change in humans over time. Continuous glucose meters (CGMs) have revolutionized diabetes care and have spared millions of diabetic patients the need for repeated self-testing by pricking their fingers every few hours. However, CGMs still suffer from major shortcomings with regard to accuracy, precision, and stability. This is primarily due to their reliance on an enzymatic detection mechanism. Current CGMs rely on the glucose oxidase enzyme to catalyze the oxidation of β-D-glucose to produce gluconic acid and hydrogen peroxide which are detected electrochemically. Unfortunately, the thermal and chemical stability and durability of enzymes are greatly affected by factors such as the pH, temperature, oxygen partial pressure, and ambient humidity level, which in turn directly affects the accuracy and analytical performance of CGMs. Thus, there is an urgent need for a different modality for real-time glucose measurements that are more accurate and robust.

One of the biological fluids that is currently under investigation as a source of biomarkers is dermal interstitial fluid or ISF. ISF is an ideal candidate for continuously monitoring biomarkers like glucose, due to the absence of clotting factors. Many platforms have already revolutionized diabetes care through enabling continuous glucose measurement via ISF. One of the newly emerged platforms as a minimally invasive extraction tool for acquiring ISF is microneedle (MN) arrays. Several MN designs have been employed for continuous glucose monitoring, including solid, hollow, and porous MNA. Hollow and porous MNA may enhance the fluid collection, but the manufacturing process and the integration with fluid collectors can be complicated. Solid MN arrays are easier to fabricate and can act as sensing transducers where arrays coated with conductive layers such as platinum, porous Au, and conductive polymer were used for continuous glucose monitoring. However, they cannot extract fluids which limit their capability for sensing. Hydrogel MN (HMN) patches are new MNs that combine the advantages of solid, hollow, and porous MNs. They are biocompatible, can extract a higher amount of ISF (9 μL vs 3μL compared to hollow MN for 100 microneedles arrays), have lower fabrication cost and higher production yield when comparing to other MNs. HMNs can uptake ISF upon insertion to the skin via diffusive force in hydrogel MNs. The insertion is generally painless and detachment has no adverse skin effect. Because of these advantages, it has been found that HMNs have potential for diagnostics where the extracted ISF is used for off-chip detection of different analytes. However, they have not yet been exploited for in situ sensing.

Herein is introduced a MN-based biosensor using swelling hydrogel for in situ, continuous, real-time, and enzyme-free glucose that has a 3D conductive structure for sensing. The MN-based biosensor is made by swellable crosslinked dopamine (DA)-hyaluronic acid (HA) hydrogel which can extract ISF effectively upon insertion to skin. A new approach was used to detect glucose where platinum (Pt) nanoparticles (NPs) were synthesized within three dimensional (3D) porous hydrogel scaffolds for non-enzymatically sensing of the glucose in ISF. The incorporation of a highly water dispersible conductive polymer, poly(3,4-ethylenedioxythiophene) polystyrene sulfonate (PEDOT:PSS) into the hydrogel network enabled boosting the electrical properties of MN, making the patch suitable for as the working electrode of the biosensor. Using the three electrode systems all consisting of MN arrays, glucose was detected in concentrations ranging from 0 to 35 mM in 1xPBS buffer solution. The sensor was also examined using an in vitro agarose hydrogel containing different concentrations of glucose (0-35 mM). The MN arrays were also tested in vivo on insulin treated diabetic rat animal models.

### 1.2 Results and Discussion

### Working Mechanism of HMN-based Glucose

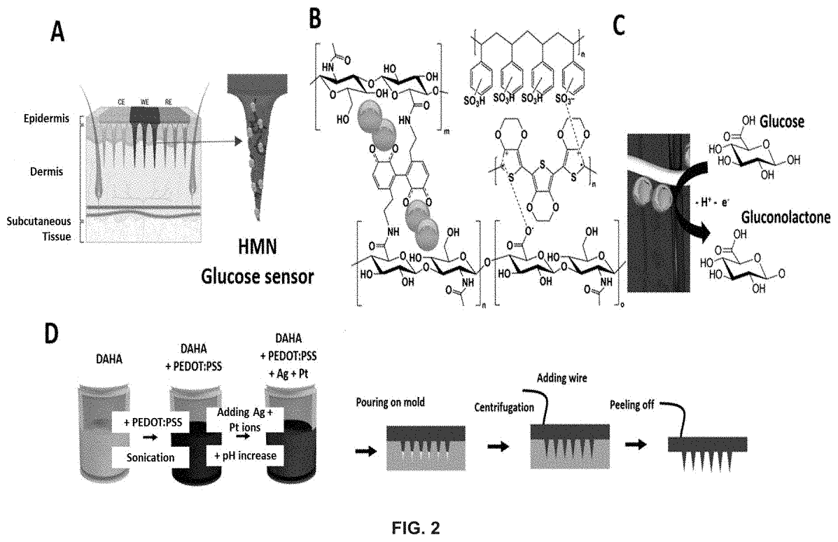

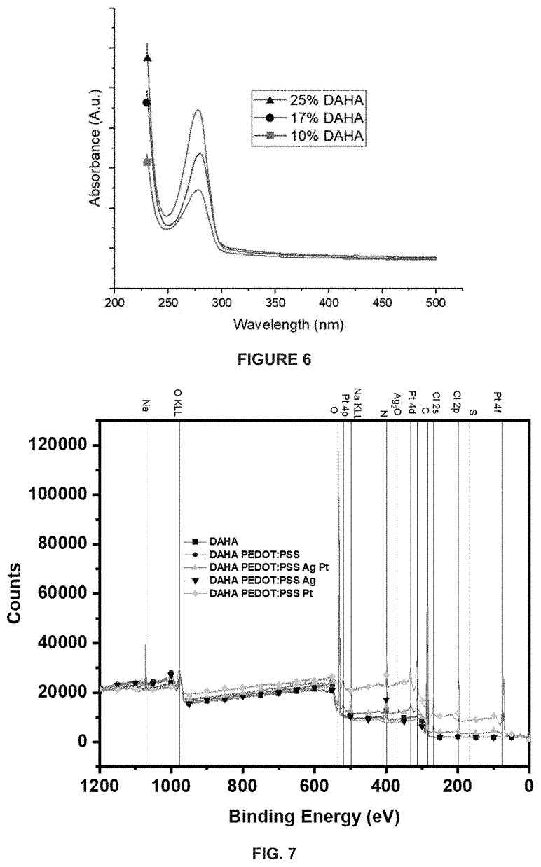

Herein, DAHA PEDOT:PSS Ag Pt HMN arrays were fabricated for glucose sensing purposes. These arrays were employed on the skin as the working electrode, along with a MN-based Ag/AgCl reference electrode and a MN Gold array as the counter electrode (FIG. 2A). To fabricate the hydrogel MN-based biosensor, first, DA-HA conjugate was synthesized using EDC/NHS coupling reaction. The conjugation of DA to HA was then investigated using ¹H nuclear magnetic resonance (NMR, FIG. 5. The ¹H NMR map shows peaks at 6.95-6.60 ppm which confirms the aromatic rings of DA. The content of DA in the polymer product was also determined with different DA conjugation rates (10%, 17%, and 25%) by measuring the absorbance at 280 nm (FIG. 6). Next, DA-HA and PEDOT:PSS 1.3 wt. % (in water) were mixed together to form a homogenous polymeric mixture (FIG. 2B). By increasing the solution pH to 8, the Carbon atoms of the benzene rings covalently bind together and form DA-DA conjugate sites resulting in the chemically crosslinked polymeric mixture. This crosslinking entraps the PEDOT:PSS polymeric chains within the hydrogel network. On the other hand, it was hypothesized that the unconjugated Carboxylate will form an ionic linkage with PEDOT positive charge of the thiophene ring. By addition of Silver(I) and Platinum(IV) solutions, Ag and Pt NPs within the Hydrogel were synthesized using the redox activity of dopamine (DA) (FIG. 2C). The fast formation of Ag NPs by DA will also improve the formation of Pt on its surface by the galvanic replacement reaction. Upon successful insertion of the 3-electrode system to the skin, the Pt NPs act as electrocatalysts to oxidize glucose, enabling non-enzymatic reaction for detecting glucose on their surface (FIG. 2C). After fabrication of both DAHA PEDOT:PSS Ag Pt hydrogel, MN-based arrays of the 3-electrode system were fabricated (FIG. 2D). Briefly, DAHA PEDOT:PSS Ag Pt solution was cast into the polydimethylsiloxane (PDMS) 10×10 MN array mold (Height: 800 μm). The loaded mold was centrifuged to remove air bubbles to ensure the hydrogel is pulled to the needle tips. Then, a copper wire was added for electrical connection and secured with UV curable epoxy. The patches were then left to dry in a nitrogen desiccator and removed from the PDMS mold once dried (see 1.3 Methods).

FIG. 2 shows a schematic representation of the Glucose sensing strategy and HMN-pH and glucose meter fabrication. FIG. 2A depicts the three-electrode set-up in the epidermal layer of the skin, showing minimal interaction between MNs and nerves. Set-up includes an Au MN-CE, an Ag/AgCl MN-RE, and DAHA PEDOT:PSS Ag Pt HMN-WE. FIG. 2B is a schematic of the composition of HMN Biosensor. The hydrogel is crosslinked by dopamine-dopamine conjugation and Catechol moieties which help the formation of Ag and Pt nanoparticles. The conductivity has been increased by incorporation of PEDOT:PSS. FIG. 2C shows the non-enzymatic reaction for detecting glucose on the surface of Platinum Nanoparticles embedded in the hydrogel network. In FIG. 2D, fabrication of the HMN-WE. Patch solution is crosslinked by NaOH and after addition of Ag and Pt ions poured onto a PDMS mold under vacuum. A Cu wire is attached to the patch and held in place by UV curable epoxy.

### Characterization of the Structure and Composition of Glucose Sensor:

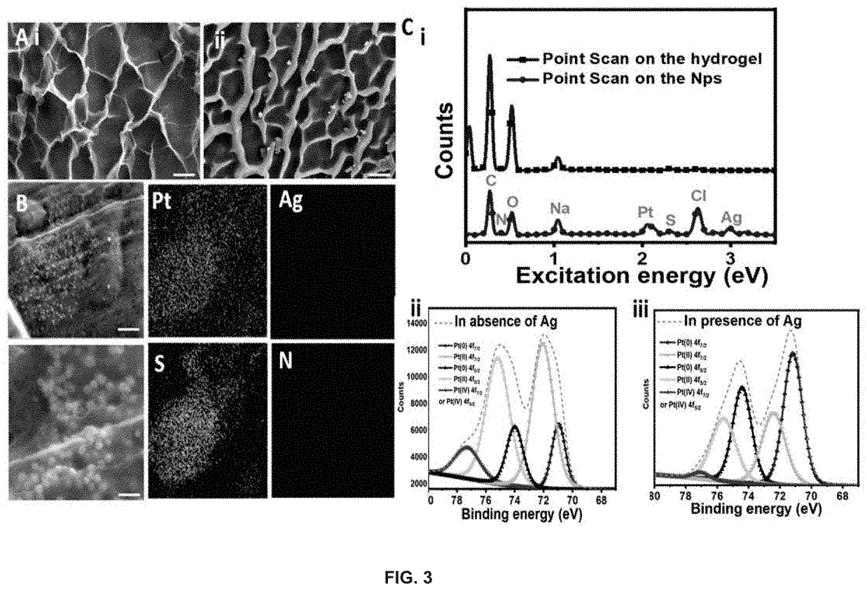

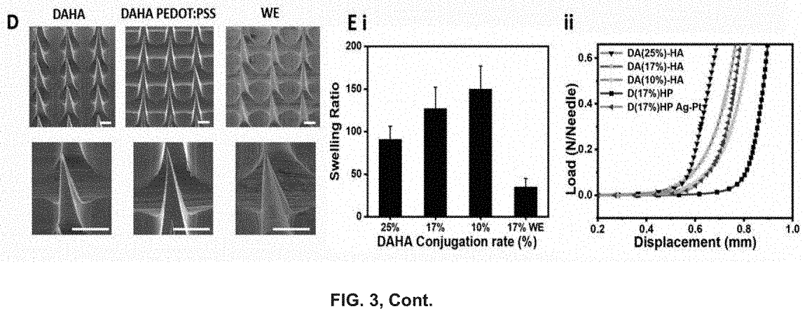

For better adhesion of Pt NPs to hydrogel network, an aim was to embed Pt NPs inside the hydrogel scaffold by in-situ reduction of Pt ions using redoxactive property of DA-HA hydrogel, where the catechol moieties of DA facilitate binding and reduction of ions. For this purpose, at first, Pt ions were added to the DAHA PEDOT: PSS hydrogel stock solution. After drying in room temperature, the dried polymeric network of DAHA PEDOT:PSS Pt was immersed in water and lyophilized to assess the successful entrapment of NPs after water uptake. The DAHA PEDOT:PSS Pt structure was investigated by the SEM images where no sign of NPs was found when the hydrogel underwent swelling (FIG. 3.Ai). For effective immobilization of Pt, the reduction of Pt ions in DAHA PEDOT:PSS hydrogel grid was done again in presence of Ag NP. In contrast to the DAHA PEDOT:PSS Pt hydrogel SEM images, the metallic nanoparticles in DAHA PEDOT:PSS Ag Pt were clearly visible after swelling (FIG. 3.Aii). The DAHA PEDOT:PSS hydrogel containing Ag and Pt NPs was then investigated by EDX mapping analysis shown in FIG. 3B. The elemental map shows the presence of Platinum, Silver on the same sites of formed NPs. Moreover, Sulfur and Nitrogen elements visible in both EDX spectrum and map suggest the presence of PEDOT:PSS and DAHA in the hydrogel, respectively. The EDX spectrum of the nanoparticles also shows significant element numbers of both Pt and Ag elements on distributed NP region in comparison to the background detected elements (FIG. 3.Ci). However, this distribution and formation of NPs are not seen in the hydrogel if Ag ions were not added. It has been found that the galvanic replacement reaction is the main factor for having a higher reduction of Pt ions to Pt nanoparticles in presence of Ag Nanoparticles. To confirm the effect of catechol and galvanic replacement reduction on Platinum Nanoparticle formation, the XPS measurement has been done (FIG. 7). The XPS spectra of Pt 4f show the concentration of Pt Metals and Pt black (PtO₂) by 60% (FIG. 3C iii) in the presence of Ag. This concentration is reduced to 30% when Ag was not added (FIG. 3C ii). Next, the SEM images of the HMN based are shown (FIG. 3D). It was observed that with a full 25% conjugation rate the WE MN arrays cannot form well. Therefore, to construct a sharp WE array, the amount of DAHA conjugation was decreased to 17%. Three different DAHA conjugation rate have also been synthesized and the swelling ratio was examined on Agarose parafilm skin model. 10% DAHA showed a higher swelling ratio compared to 25% and 17% due to less crosslinking degree. After the addition of the nanoparticles, the patch undergoes a lower swelling ratio. The low swelling property of WE could be attributed to the presence of nanoparticles. The robustness of the different HMN compositions was also tested by compression test using a DMA machine. The 25% DAHA shows a higher slope than 17% and 10%. Also, the addition of PEDOT:PSS to DAHA increases the flexibility of the sensor. However, introducing platinum and silver nanoparticles improves the robustness of the sensor.

FIG. 3A shows (i) is an FESEM image of DAHA PEDOT:PSS Pt hydrogel after swelling where no traces of NPs were found. (ii) FESEM image of DAHA PEDOT:PSS Ag Pt hydrogel after swelling and the presence of nanoparticles. (Scalebar: 30 μm) FIG. 3B shows EDX mapping on the region that NPs are abundant. The maps show high amount of Pt, Ag, S, and N atoms in the vicinity of NPs. (Scalebar: 10 μm). FIG. 3C shows (i) EDX analysis of a single Np on hydrogel grid and comparison with the surface of hydrogel, (ii) XPS analysis of platinum 4f orbital for DAHA PEDOT:PSS Pt (iii) XPS analysis of platinum 4f orbital for DAHA PEDOT:PSS Ag Pt hydrogel. FIG. 3D shows SEM images of DAHA, DAHA PEDOT:PSS, DAHA PEDOT:PSS Ag Pt (WE), and zoomed-in of their microneedle. (Scalebar: 300pm). FIG. 3E) shows (i) Swelling ratio of DAHA on Agarose-parafilm skin model for 25%, 17%, 10% DAHA and 17% DAHA PEDOT Ag Pt (WE). (ii) Plot of Compression tests of the different composition of Hydrogel microneedle arrays (where DHP is DAHA PEDOT:PSS).

### Sensing Characterization of the HMN Glucose Sensor:

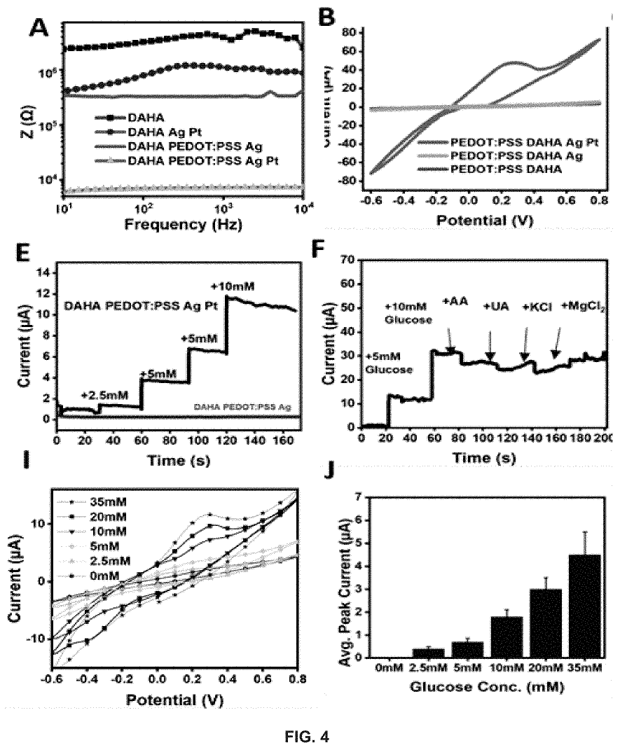

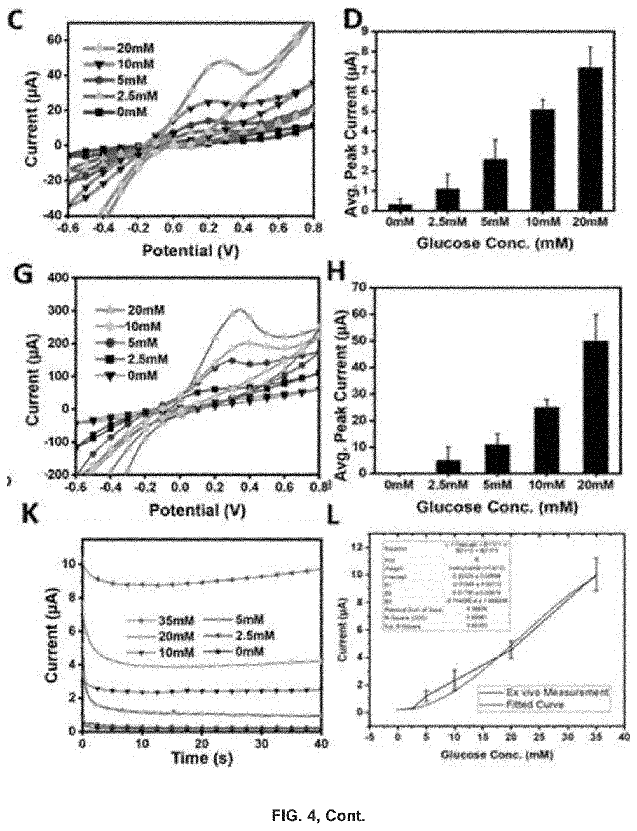

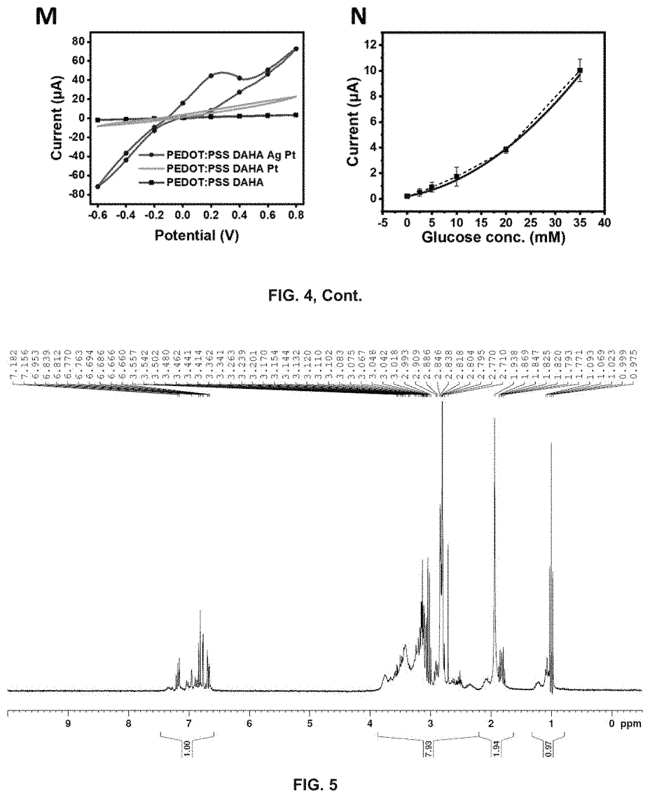

The electrochemical impedance spectroscopy of DAHA, DAHA Ag Pt, and DAHA PEDOT:PSS(5%) Ag Pt hydrogel thin films in 1x PBS buffer solution have been employed to study the suitability of the hydrogel as the electrochemical biosensor. DAHA PEDOT:PSS Ag Pt hydrogel thin films showed higher conductivity than other hydrogels thin films (FIG. 4A). Upon successful fabrication of DAHA PEDOT:PSS Ag Pt hydrogel, the glucose sensing was investigated, where cyclic voltammetry (CV) scanning was performed in 1xPBS solution containing varying glucose concentrations (0 mM-2.5 mM-5 mM-10 mM-20 mM). Here, DAHA PEDOT:PSS Pt Ag thin films were used as the working electrode, commercial Ag/AgCl reference electrode and a commercial Pt wire as the counter electrode. A peak was found for each CV measurement around 0.2 to 0.3 V which corresponds to glucose oxidation on the surface of Pt NPs (FIG. 4B and 4C). The DAHA PEDOT:PSS hydrogel thin film samples with or without Ag or Pt NPs were used to detect 20 mM glucose in 1xPBS solution using CV measurement. A redox peak was found in CV measurement around 0.2-0.3 V where both Ag and Pt NPs were incorporated while for the other two samples no peak was observed (FIG. 4M), Next, chronoamperometry scanning was used for real-time detection of rising concentrations of glucose (FIG. 4E). The selectivity of the sensor for glucose detection was also examined in the presence of common interfering agents in ISF, i.e., ascorbic acid, uric acid, KCl and MgCl₂ (FIG. 4F). The concentrations of glucose used were 5 mM and 10 mM and the concentration of the interferents used in the investigation was 10-15-fold higher than the normal physiological level. FIG. 4F shows that the addition of any of these common interferents had a negligible effect on the glucose response. Glucose sensing was then investigated by the three MN electrode system (FIG. 2A) applied on agarose hydrogel containing various glucose concentrations and CV scanning was performed (FIG. 4G and 4H). In the next step, ex vivo experiments were carried out, in order to determine the sensing capability of the MN sensor. To this end, porcine ear skins were incubated into different concentrations of glucose (0 mM, 2.5 mM, 5 mM, 10 mM, 20 mM, and 35 mM) in 1X PBS and overnight. Similar to previous experiments, CV scanning was performed and the ex vivo glucose measurement result is depicted in FIG. 4G and 4H. The glucose concentrated porcine skins were then studied by Chronoamperometry measurements at 0.4 V for 45 seconds. The resultant chronoamperometry curves are presented in FIG. 4K. FIG. 4L and 4N show the calibration curve of current against glucose concentrations (R²=0.96).

FIG. 4: shows in vitro characterization of a hydrogel microneedle-based glucose biosensor. FIG. 4A shows EIS measurements of DAHA, DAHA Ag Pt, DAHA PEDOT:PSS (5%) Ag Pt. FIG. 4B shows CV measurement for a comparison between the hydrogel microneedle-based glucose biosensor film (DAHA PEDOT:PSS Ag Pt) and DAHA PEDOT:PSS film in 1xPBS buffer containing 20 mM glucose at scan rate of 20 mV/s. FIG. 4C shows CV measurement of hydrogel microneedle-based glucose biosensor films in 1xPBS buffer containing glucose concentrations equal to 0, 2.5, 5, 10, 20 mM at scan rate of 20 mV/s. FIG. 4D shows the corresponding Bar graph plot of CV measurement of 1xPBS buffer containing glucose concentrations equal to 0, 2.5, 5, 10, 20 mM, FIG. 4M shows DAHA PEDOT:PSS hydrogel thin film samples with or without Ag or Pt NPs were used to detect 20 mM glucose in 1xPBS solution using CV measurement. FIG. 4E shows Chronoamperometry Measurement at 0.4 V in 1xPBS and step-wise addition of 50 μl, 100 μl, 100 μl, and 200 μl every 30 second (Total glucose concentrations equal to 2.5, 7,5, 12.5, and 22.5 mM). FIG. 4F shows the selectivity test of interfering agents by chronoamperometry measurement at 0.4 V in 1xPBS and step-wise addition of 0.5 mM ascorbic acid, 0.5 mM uric acid, 0.5 mM KCl and 0.5 mM MgCl₂. FIG. 4G shows CV measurement by HMN biosensor on agar-parafilm containing glucose concentrations equal to 0, 2.5, 5, 10, and 20 mM at scan rate of 50 mV/s. FIG. 4H shows the corresponding Bar graph plot of CV measurement of 1xPBS Agarose gel containing glucose. FIG. 41 shows a CV measurement by HMN biosensor on porcine skin incubated with glucose concentrations equal to 0, 2.5, 5, 10, 20, and 35 mM at scan rate of 50 mV/s. FIG. 4J shows the corresponding Bar graph plot of CV measurement of porcine skin incubated with various glucose concentrations. FIG. 4K shows Chronoamperometry measurement by HMN biosensor on porcine skin incubated with glucose concentrations equal to 0, 2.5, 5, 10, 20, and 35 mM at 0.4V for 45 seconds. FIG. 4L and 4N shows the corresponding calibration plot of the chronoamperometry measurement.

### 1.3 Materials and Methods for HMN Glucose Sensor

### Materials

Hyaluronic acid (HA, MW 200-400 KDA, AquaJuve™ Plus 2040) was purchased from Joyvo Co., Ltd (China). Dopamine hydrochloride (DA), N-(3 Dimethylaminopropyl)-N′-ethylcarbodiimidev (EDC), N-Hydroxysuccinimide (NHS), Sodium hexachloroplatinate(IV) hexahydrate, Ascorbic Acid (AA), Uric Acid (UA), Magnesium Chloride (MgCl₂), Potassium Chloride (KCl), Ethylene Glycol (EG), Silver nitrate (AgNO₃), UV curable resin (55 cp), Silver/Silver Chloride (60/40) paste for screen printing, Agarose Type I-A (A0169) were purchased from Sigma Aldrich (Canada). PEDOT:PSS (PH 1000—Heraeus) was purchased from Clevios™.

### DAHA Fabrication Protocol:

2 g HA was dissolved in 200 mL of Milli-Q water overnight. 970 mg EDC and 583 mg NHS were then added to the HA solution. Next, 1 g Dopamine hydrochloride was added and completely dissolved after the pH was changed to 5 using 1M hydrochloric Acid. After overnight reaction, when the solution color turned to dark brown, it was dialyzed at room temperature in Dialysis tubing cellulose membrane (Sigma Aldrich, MWCO=4,000) for 3 days at pH 4.5. The solution was transferred to falcon tubes and freeze-dried, DAHA conjugation was confirmed by NMR and ultraviolet-visible spectroscopy (BioTek, Synergy H1). The dopamine-HA conjugation was determined by UV absorbance at 280 nm.

### Incorporation of PEDOTPSS and In-Situ Synthesis of Silver and Platinum Nanoparticles:

380 μL PEDOT:PSS and 2 mL DI water were added to a 20 ml glass vial. 38 μl Ethylene Glycol was added to the solution and sonicated for 5 minutes. Next, 100 mg of DAHA was added to the solution and held in the desiccator for 3 hours to be completely dissolved. 1M silver nitrate and 1M Platinum(IV) sodium chloride stock solutions by dissolving 1 millimole of each in 1 ml DI water were prepared. The pH of the solution was adjusted to 7.5 using NaOH and 40 μl from prepared silver nitrate stock solution was added to vial and stirred with spatula until the solution color turned to gray. Next, 24 μl from the platinum stock solution was added and stirred with spatula for 1 minute. The solution was whether transferred to MN mold for MN patch formation or drop casted on glass slides for the film fabrication.

### Preparation of MN Three-Electrode Sensor:

In order to make working electrode MN arrays, the solution containing Silver and Platinum nanoparticles as explained in previous step were transferred on the 10×10 MN mold, To remove the bubbles trapped inside the MN tip, vacuum has been done at 25 mmHg for 2 minutes. After the bubbles were successfully removed, a conventional electrical wire was left on the solution and let it dry at room temperature for 24 hours. The WE MN arrays were peeled off easily from the mold. In order to make sharp and robust counter and reference electrode microneedle arrays for effective penetration, a UV cured epoxy solution was added on top of MN mold. The solution on the mold was vacuumed to remove the bubbles and then it was cured by UV for 2 minutes. The solid epoxy MN arrays were peeled off from the mold. Next, to form the Counter electrode Chrome and Gold (Cr 10 nm/Au 75 nm) were deposited on the epoxy microneedles by thermal evaporation (Intevac. Canada). The reference electrode was fabricated by deep coating of epoxy microneedles to silver/silver chloride solution and dried by nitrogen spray gun. Wires were attached to the back of counter and reference electrode MN using silver conductive epoxy (MG Chemicals).

### Swelling Experiments on Agarose Skin Mimicking Model:

Agarose gel was prepared by a mixture of 140 mg agarose and 10 mL DI water (1.4%). To make the skin mimicking model, the top surface of agarose gel was covered by a layer of parafilm The MN patches were punched through agarose by fingertip and kept on the skin model using another layer of parafilm for 10 min. The hydrogel MN patches weight were measured before and after being inserted on skin model. The swelling ratios were measured as explained elsewhere.

### Characterization of MN Arrays:

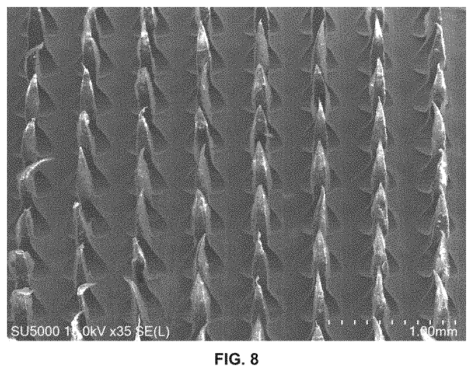

The hydrogel MN were kept inside the DI water overnight for maximum swelling. Next, swelled patches were transferred to liquid nitrogen. The frozen hydrogels were freeze dried. Next, the surface morphology of hydrogel MN patches was characterized by scanning electron microscopy (SEM, Zeiss Ultra Plus, e.g. see FIG. 8). The surface element analysis was performed using EDX (Oxford Instrument).

Electrochemical Sensing by film and MN Patch:

A conventional three-electrode electrochemical cell and a Potentiostat (Palmsens4) were used for the cyclic voltammetry and chronoamperometry measurement of the films in 1x PBS buffer solution containing different concentrations of Glucose, AA, AU, KCl, MgCl₂. Each film (5 mm×15 mm) was attached to working electrode clamps as working electrode and a commercial Ag/AgCl reference electrode and a commercial Pt counter electrode were used. The CV result of sensing glucose at 0, 2.5, 5, 10, 20 mM in 1x PBS solutions (pH 7.4) were done at the scanning of 20 mV/S and a potential range of −0.6 to 1 V. For chronoamperometry measurement the potential was set to −0.4 V. For measuring the response of glucose addition, a stock of 1M glucose was prepared and it was added step-wisely every 30 second to 1x PBS buffer solution to make 2.5, 7.5, 12.5, 22.5 mM glucose concentrations. The hydrogel MN sensor arrays were penetrated through the skin model with different concentration of glucose (0-20 mM). The conditions are the same as CV measurement in the buffer solution.

### Example 2: A Hydrogel-Based Microneedle Platform for Real-Time pH Measurement in Live Animals

Reference numbering begins anew in Example 2, with references listed in section 2.5.

### 2.1 Introduction

Physiological pH monitoring is of clinical importance as it provides information regarding the body's regulatory systems. A slight change in physiological pH can have effects on cellular, tissue, and organ functions, and can thus be an indicator for early disease detection. For example, monitoring cellular pH can be important for cancer regulation, monitoring tissue pH can be important in the diagnosis of diseases such as ischemic [3], and monitoring the pH of organs is valuable in screening for conditions such as renal failure and acidosis [3].

The last decade has brought about advances in wearable sensors, so that they are currently used by 24% of the American adult population. Wearable sensors have a growing importance as they aid in the monitoring of health conditions and provide a means for earlier disease detection, all with the benefits of low cost, high performance, limited access to doctors, and miniaturization. pH can be monitored by wearable sensors through the analysis of bio-fluids including urine, saliva, sweat, interstitial fluid (ISF) and blood. Of the existing wearable biosensors for pH detection, the majority use saliva and sweat as the bio-fluid of choice.

Saliva-based biosensors measure pH to monitor oral health, dental erosion, tooth decay, plaque build-up, and gingivitis. For example, a sensor that utilizes Silver/Silver Chloride (Ag/AgCl) and Antimony/Antimony Trioxide (Sb/Sb₂O₃) electrodes was reported for pH measurement based on the change in the electrode potential. The sensor is mounted on a mouth guard and passes information using an radio frequency transponder. Limitations with salivary sensors include low measurement sensitivity due to plaque and biofilm build-up and a large sensor footprint. In addition, saliva is less readily available and contains some charged proteins that are not present in other bio-fluids. The rough surfaces in the mouth and teeth are also not ideal for the formation of a good contact with the sensor.

Sweat-based biosensors measure pH to screen for diseases such as diabetes and cystic fibrosis. Textile-based pH sensors utilize conductive fibers such as cellulose-polyester blend, cotton yarn combined with polyaniline and poly(3,4-ethylenedioxythiophene) polystyrene sulfonate (PEDOT: PSS) (two conductive polymers) and carbon nanotubes to effectively absorb sweat. Similarly, bandage-based sensors integrate the electrodes onto a regular bandage surface. Finally, flexible sensors monitor the pH of sweat by utilizing flexible polymeric materials for the creation of a skin-conforming electrode mount [15]. Despite the ease of wear for textile sensors, they can undergo mechanical and chemical damage once washed. Sweat-based sensors also pose limitations due to low sweat rate, sample evaporation, and bacterial contamination from skin surfaces. In order to validate sweat-based sensors, the concentration of analytes in sweat must correlate to those in blood, and sensors are not able to adequately collect the volumes required.

Other limitations with current wearable sensors include resiliency, long-term stability, biocompatibility, repeatability, reproducibility, and validation against blood analyte levels. In some cases where microfabrication and lithographic techniques are required, cost may be a limitation to sensor fabrication. In addition to device restrictions, saliva and sweat have fewer biomarkers present as well as lower concentrations.

The use of ISF in wearable sensors has caught attention as it holds an 83% protein similarity to blood plasma, lacks dotting agents found in blood, holds increased sensitivity to acid-base balance, and can be collected in a minimally-invasive manner [5], [20], [21]. ISF is present in the interstitial space under the dermal layer of the skin, making it very abundant and easily accessible [20].

Microneedle (MN) based transdermal biosensing is a minimally-invasive approach to collect and measure ISF. The fast healing of the injection site, and a decreased chance of bacterial contamination of the injection site are other advantages. MN-based biosensor technology can be classified in two categories: on-needle and off-needle sensing. The reported on-needle biosensors utilize solid and coated MNs which act as the device electrodes for electrochemical measurement. The direct interaction between analytes and electrodes creates a current response that can be measured. These electrodes are typically made of solid metal or metallic coatings, creating biocompatibility issues with the electrodes as well as high fabrication costs [3], [5]. Off-needle MN biosensors directly extract ISF for post-processing and analysis. In these applications, hollow glass, silicon, or metal MNs are used to extract ISF using capillary force [21]. These can be disadvantageous as they are limited to low extraction volumes [21] and are not biocompatible [21]. Hydrogel MNs (HMNs) are an alternative to hollow needles since they extract ISF into their swellable porous matrix [20], [21]. They are an emerging technology that is simple, efficient, biocompatible, biodegradable, creates minimal tissue damage, and have ease of fabrication [20], [21].

Currently, Zheng developed HMNs for the in-situ detection of glucose by integrating their HMNs with an external glucose sensor [20]. Their hydrogel MNs consisted of a methacrylated hyaluronic acid (MeHA) network with maltose. They were able to achieve quick ISF extraction due MeHA's hydro affinity and the increase in osmotic pressure from maltose [20]. The authors are unaware of any HMN patches for pH detection, however pH-sensitive hydrogels have been developed.

Herein is disclosed a HMN biosensor for in-situ, real-time pH measurements in ISF, that is capable of a clinically relevant detection range at a pH of about 3 to about 8. The HMN-pH meter is the first MN patch composed of a dopamine (DA) conjugated hyaluronic acid (HA) hydrogel network which can be easily crosslinked by adjusting the patchs' pH. The HMN patch is also laced with poly(3,4-ethylenedioxythiophene) polystyrene sulfonate (PEDOT:PSS) to increase conductivity, making the patch suitable as the working electrode (WE) of the sensor. The catechol-quinone chemistry intrinsic to dopamine is utilized as a sensing mechanism, allowing for the measurement of a current response as a function of pH level. The sensor bridges the gap between traditional metallic electrochemical biosensors and the direct extraction of ISF, overcoming many of the associated limitations mentioned with wearable sensors. The hydrogel network is biocompatible and has a swelling rate of 250%, allowing for a higher accessible ISF sampling volume. There is also no post-processing of ISF required, and the catechol-quinone chemistry is selective to Hydrogen ions (H¹), meaning measurements are not affected by crosstalk from other ions present in the ISF.

### 2.2 Results & Discussion

### pH Sensing Strategy and HMN-pH Meter Fabrication

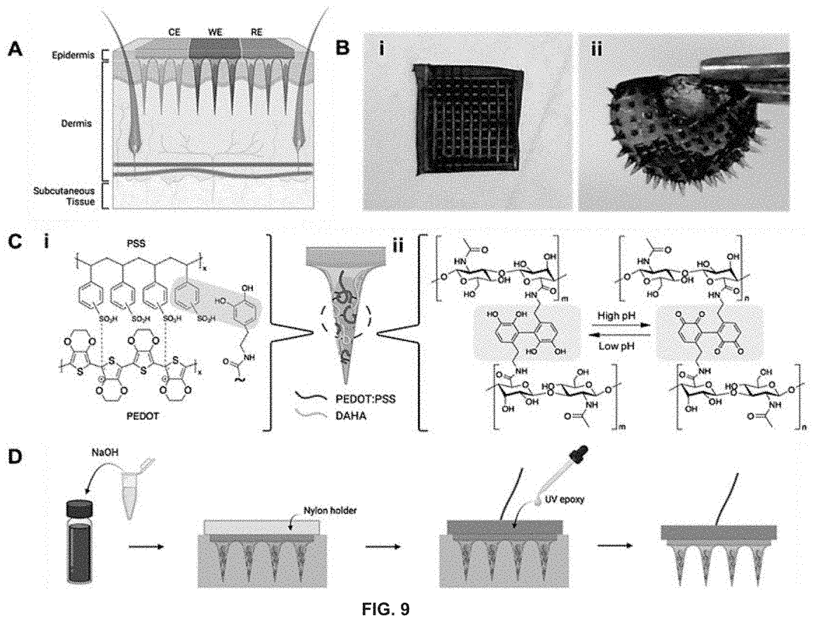

FIG. 9 shows a schematic representation of the in-situ pH sensing strategy and HMN-pH meter fabrication. FIG. 9A exemplifies a three-electrode set-up in the epidermal layer of the skin, showing minimal interaction between MNs and nerves. Set-up includes an Au MN-CE, an Ag/AgCl MN-RE, and a PEDOT:PSS HMN-WE. FIG. 9B shows PEDOT:PSS HMN-WE before (i) and after (ii) swelling from a 10 minute penetration through parafilm-covered agarose. HMN-WE have a 10×10 array of needles that are 800 μm tall. FIG. 9C shows chemical sensing mechanism of the HMN-WE for pH detection and signal transduction. PEDOT:PSS and DA-HA form a hydrophilic hydrogel network which can expand and uptake ISF. (i) Integration of PEDOT:PSS in the hydrogel network through pi-pi stacking with DA. (ii) pH sensitivity of DA. At low pH, DA will have a catechol structure, and a quinone structure at high pH. Phenol-phenol linkages as well as u-u stacking is observed between DA-HA structures. FIG. 9D shows fabrication of the HMN-WE. Patch solution is crosslinked by NaOH and poured onto a PDMS mold under vacuum, or centrifuged, to remove air bubbles. A Cu wire is attached to the patch and held in place by UV epoxy.

The exemplified HMN pH-meter consists of a three-electrode set-up that can directly measure the H⁺ concentration in ISF via the catechol-quinone chemistry of DA (FIG. 9A). The three electrodes include HMNs as the working electrode (HMN-WE), Ag/AgCl MNs as the reference electrode (MN-RE), and gold (Au) MNs as the counter electrode (MN-CE). The HMN-WE consists of a 10×10 array of 800 μm tall needles with a pyramidal geometric design (FIG. 9Bi) capable of swelling (FIG. 9Bii). Such needle designs allow the HMNs to pierce the epidermis and extract ISF without reaching the nerve fibers or blood vessels in the dermal layer, providing a minimally invasive and virtually painless method to directly measure ISF pH levels. The MN-RE of the pH meter was fabricated by coating an epoxy MN patch with Ag/AgCl paste, and MN-CE was fabricated by sputtering an Au layer on an epoxy MN patch.

The WE comprises of HMNs made of a DA-HA hydrogel network laced with PEDOT:PSS. The DA-HA hydrogel is porous and can facilitate the uptake of ISF, while PEDOT:PSS enhances WE conductivity allowing the HMN-WE to have on-needle sensing without the integration to an external sensing device [33]. PEDOT:PSS creates a polymer blend with DA-HA through physical entanglement and π-π stacking (FIG. 9Ci & 9Cii).

Without wishing to be bound by any one theory, it is noted that DA-HA can detect and selectively sense changes in H⁺ concentration due to the effect of pH on the catechol-quinone structure (FIG. 9Bii). At pH values below 6, DA is reduced to the catechol form, and at pH values above 9, DA is oxidized to the quinone form. The oxidation and reduction of DA creates a measurable change in current through the WE. This is beneficial, as it allows the selective sensing of H⁺ in the ISF, minimizing background noise or false positives from other cations that may be present. Phenol-phenol linkages between DA also add to the hydrogel cross-linking which increases the uptake of ISF.

A HMN-WE was created by cross-linking a solution of PEDOT:PSS with DA-HA and loading it into a PDMS mold (FIG. 9D). The loaded mold was centrifuged to remove air bubbles and to ensure the hydrogel is pulled to the needle tips. Then, a copper wire was added for electrical connection and secured with UV epoxy. The patches were then left to dry in a nitrogen desiccator and removed from the PDMS mold once dry (see 2.4 Methods).

### HMN Characterization and Optimization

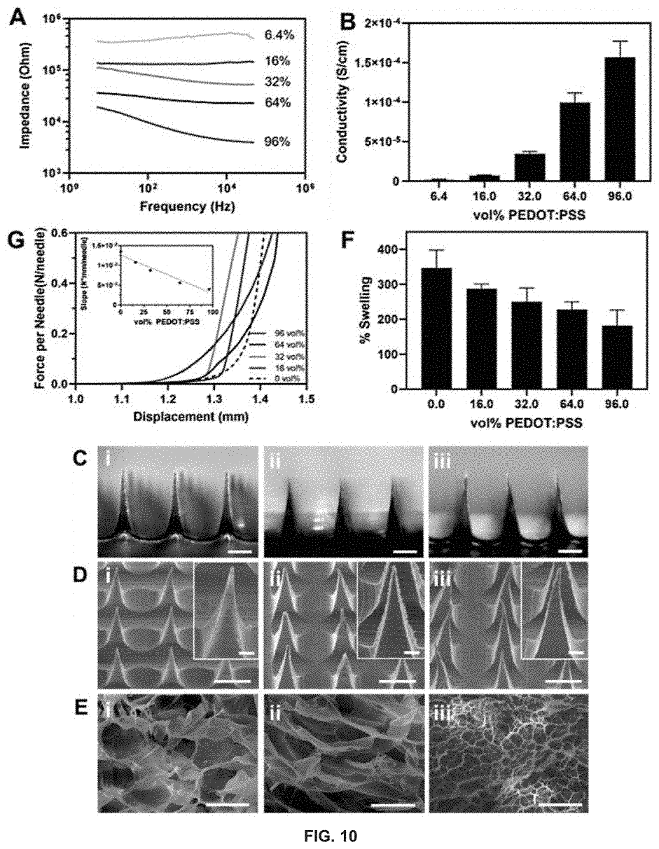

FIG. 10: shows the characterization and optimization of the HMN polymer and needles. FIG. 10A shows EIS for thin films of the HMN patch mixture using Ag/AgCl

RE, Pt CE, and lx PBS as the buffer solution. FIG. 10B shows 4-point probe measurements for thin films of the HMN patch mixture. FIG. 10C shows 2D optical microscope images of (i) 0 vol % (ii) 16 vol % and (iii) 32 vol % PEDOT:PSS HMN WE (vol % relative to total liquid composition) (scale is 300 μm). FIG. 10D shows 3D SEM images of (i) 0 vol % (ii) 16 vol % and (iii) 32 vol % PEDOT:PSS HMN WE (vol % relative to total liquid composition) (scale is 200 μm). Insets show an enlarged view of a singular HMN (scale is 50 μm). FIG. 10E shows SEM images of the porosity of (i) 0 vol % (ii) 16 vol % and (iii) 32 vol % PEDOT:PSS HMN network (vol % relative to total liquid composition) (scale is 50 μm). FIG. 10F shows swelling ratios for various PEDOT:PSS concentrations in HMN patch mixture after 10 min insertion into agarose pads. FIG. 10G shows mechanical compression test for HMN-WE. Inset shows the slope of the compression curve for varying concentrations of PEDOT:PSS. An R² value of 0.9551 indicating a good goodness of fit for the model.

Since PEDOT:PSS allows for the HMNs to be conductive and act as the WE of pH-meter, the amount of PEDOT:PSS should be sufficient to ensure good conductivity, while still maintaining needle integrity for skin penetration.

First, the conductivity levels of different PEDOT:PSS concentrations were assessed in the patch solution, Electrical impedance spectroscopy (EIS) (FIG. 10A) and 4-point probe measurements (FIG. 10B) were conducted for thin film samples of varying PEDOT:PSS concentrations (See 2.4 Methods). EIS showed that impedance decreases as PEDOT:PSS concentration increases, and 4-point probe data compliments this trend, showing an increase in conductivity with an increase in PEDOT:PSS concentration. From this data, it can be concluded that increasing PEDOT:PSS concentration allows for good HMN-WE conductivity.

Optical characterization of the needles was then performed, to ensure that the addition of PEDOT:PSS does not interfere with needle integrity. Optical images of the two dimensional (2D) needle arrays (FIG. 10C) as well as scanning electron microscopy (SEM) images of three dimensional (3D) needle arrays (FIG. 10D) were analyzed. Increasing the PEDOT:PSS concentration creates needles with a less ridged structure, and introduces more flexibility into the patches which is not favorable for skin penetration. In addition, 64 vol % and 96 vol % PEDOT:PSS solutions may not have enough structural integrity, as needles have been observed to be distorted after being pulled apart from the PDMS mold (where vol % is relative to total liquid composition).

Next, the effect of PEDOT:PSS on film porosity and HMN swelling capability was assessed, Thin films were made from 0, 16, and 32 vol % PEDOT:PSS patch solutions (vol % relative to total liquid composition) and were allowed to swell in water for 10 min. The films were then snap frozen with liquid nitrogen and freeze dried for 48 hours (See 2.4 Methods). SEM images were taken to show the film porosity after swelling (FIG. 10E), showing that the porosity decreases as PEDOT:PSS concentration increases. This is important, as an increased pore size allows for a larger ISF uptake volume. To further investigate the effect of PEDOT:PSS on the ISF uptake by HMN patches, experiments were performed to characterize the swelling capability. HMN patches were applied through an agarose hydrogel (mimicking the skin environment) for 10 min where the patch's weight was measured before and after application (see 2.4 Methods). FIG. 10F shows that decreasing PEDOT:PSS concentration increases swelling, and thus allows for a larger volume of ISF to be measured.

Finally, mechanical strength testing was completed (FIG. 10G) to determine whether the MN patches have enough compressive strength to effectively pierce the epidermis. Force vs. displacement plots were obtained showing the load force sustained by a single MN before its fracture point (sharp increase in force vs. displacement plot). The fracture force associated with each MN's fracture force shows a decreasing linear relationship with respect to increasing PEDOT:PSS concentration (FIG. 10G inset). This notably indicates that decreasing PEDOT:PSS concentration also decreases the MN's flexibility, which increases the MN's ability to successfully penetrate the epidermal layer.

According to the above measurements, it was determined that the 32 vol % PEDOT:PSS patch concentration presents a good balance between conductivity and needle integrity (vol % relative to total liquid composition). The 32 vol % PEDOT:PSS patches have enough strength to penetrate the epidermis (0.13 N/needle), are able to swell to 250% their original size, present good conductivity (3.46 E-05 S/cm), and have a low impedance (53 kΩ). In the following experiments, 32 vol % PEDOT: PSS was used to fabricate the WE of the HMN-pH meter.

Ex-vivo pH Measurement using a Skin Model

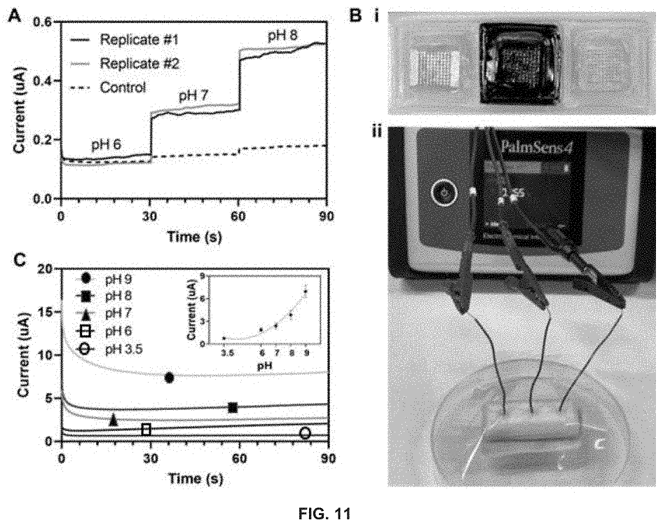

FIG. 11 shows in vitro and ex vivo characterization of pH sensing for the three-electrode system. FIG. 11Aa shows pH step-increase for a 32 vol % PEDOT:PSS HMN thin film after successive additions of 1M NaOH at 0.4 V. Ag/AgCl CE and Pt RE used (vol % relative to total liquid composition). FIG. 11B shows a three-electrode set-up from a (i) top view and (ii) side view on porcine skin, attached to the potentiostat. FIG. 11C shows an ex-vivo amperometric response of 32 vol % HMN-WE at 0.4V of the three-electrode system on pH equilibrated porcine skin. Inset shows corresponding pH calibration plot, with R²=0.9728 for goodness of fit.

Having demonstrated that a conductive HMN patch can be fabricated by addition of PEDOT:PSS, a series of experiments were conducted to investigate the HMN's pH measuring capability, pH is a measure of H⁺ ions (as seen in Equation 1) and can be quantified with the DA-based pH meter. At lower pH, due to the increase of H⁺ ions in the system, DA will be reduced to its catechol form. Comparably at higher pH, DA will be oxidized to its quinone form. On the other hand, the concentration of ions in a solution affects its ability to conduct electricity. As denoted by Equation 2, conductivity (σ) is directly proportional to current (I). Therefore, it is reasonably deduced that current should increase as DA gets oxidized.

$$pH = - log [ {H}^{+} ] \qquad ( 1 )$$

$$σ = \frac{lI}{AV} \qquad ( 2 )$$

To confirm this, a pH step increase was completed (FIG. 11A). 32 vol %, PEDOT:PSS thin films were used as the WE and were immersed in a pH-controlled PBS solution (vol % relative to total liquid composition; See Methods). It was observed that the current increases as pH increases, with an approximate 2-fold increase between pH 6, 7, and 8, while no increase in current was seen in the control sample with no pH increase.

Next_{;} the HMN's ability to detect pH changes in vitro was assessed on pH-controlled agarose hydrogels. A clear increase in current was observed with increasing pH levels. Next, the HMN three-electrode system (FIG. 11Bi) was mounted on a piece of porcine skin and connected to the potentiostat (FIG. 11Bii) for ex vivo measurements. The porcine skin was equilibrated in a pH buffer at 4° C. for 12 hours prior to the measurements (See Methods). Chronoamperometry measurements were then carried out on skins that were equilibrated to a clinically relevant pH range of 3.5, 6, 8, and 9 (FIG. 11C), and a calibration curve was created (FIG. 11C inset). It is noted that the measured current increases as pH increases, indicating that the HMN-WE is capable of on-needle detection and differentiation of varying pH levels without any post-processing required. One note of interest is that 10-fold higher current is measured in the porcine skin compared to in agarose pads for the same pH levels. Without wishing to be bound by any one theory, this has been attributed to additional ions present in the skin ISF.

### 2.3 Outlook

Conventional MNs have been extensively reported and applied towards a very specific group of applications. Hydrogel forming microneedles with the added ability to electrically sense biomolecules in real-time is an area yet to be explored. The application of such an amalgamation of a diagnostic and therapeutic system is endless, especially if this can be transformed into a wearable device.

Herein was disclosed the first DA-HA based HMN patch that is capable of on-needle detection with no post-processing required. Using DA-HA mixed with PEDOT:PSS a swellable and conductive HMN patch was created that can provide real-time pH measurements. It has been shown that the catechol-quinone chemistry intrinsic to DA is selective to H⁺ which allows for high sensor specificity. Moreover, a comprehensive characterization of the PEDOT:PSS HMN patches has been discussed, showing that PEDOT:PSS provides conductivity while maintaining the structural integrity, swelling, and mechanical strength of the HMNs. Ex vivo skin experiments indicate that the HMN-WE is capable of on-needle detection and differentiation of a clinically relevant pH range without any post-processing required.

### 2.4 Methods

### Chemicals and Materials.

Sodium Hyaluronate Hyaluronic Acid (HA) was purchased through Bloomage Biotech (Hybloom™ Low Molecular Weight Sodium Hyaluronate (HA-TLM)). Dopamine hydrochloride (2-(3,4-Dihydroxyphenyl)ethylamine hydrochloride), 1-ethyl-3-(3-dimethylaminopropyl)carbodiimide (EDC) hydrochloride, N-hydroxysuccinimide (NHS), Dialysis tubing cellulose membrane, Monobasic potassium phosphate (KH₂PO₄) and Potassium hydrogen phthalate were purchased from Sigma-Aldrich. Milli-Q water was used as deionized water in hydrogel synthesis and experiments. PEDOT:PSS (1.3 w.t. %) was obtained from Ossila, and Microneedle molds were procured via Micropoint Inc (Singapore).

### Synthesis of Dopamine-Conjugated Hyaluronic Acid (DA-HA),

The DA-HA was synthesized by carbodiimide coupling chemistry [36]. Briefly, two grams of Sodium Hyaluronate were fully dissolved in 100 mL of Milli-Q deionized water at room temperature. 970 mg of EDC and 583 mg of NHS were gradually added into the HA solution. After 30 mins, 1 g of dopamine was added to the solution and the pH of the mixture was adjusted to 5-6. The solution was then stirred for 9 hours where a certain change in color of the solution was observed due to the addition of dopamine from clear to dark brown. The blend was then added into the dialysis tubing which is a proven method for purification of HA [37]. The process of Dialysis/Purification may be varied, however, the best results were observed upon leaving the dialysis tubes inside an acidic media for 3 days. The DA-HA solution was then poured into 50 mL Eppendorf tubes and allowed to freeze at −20° C. overnight. The frozen mixture was then put into a freeze dryer for 3 days. The chemical structure of the resulted polymer was characterized by H-nuclear magnetic resonance (NMR) (Ascend 400 MHz).

Used herein: 0, 1, 2.5, 5, 10, and 15 wt % PEDOT:PSS (where wt % is relative to DAHA, as used in Examples 2 and 3) is respectively equivalent to 0, 6.4, 16, 32, 64, and 96 vol % PEDOT:PSS (where vol % is relative to total liquid composition, as used in Example 2).

### Amalgamation of PEDOT:PSS in DA-HA.

The DA-HA acquired from the freeze dryer is combined with an electrically conductive media called PEDOT:PSS. The concentration of DA-HA for the hydrogel is set to 50 mg per 1 mL of solution; or is set to 50 mg per 0.6 mL of solution. With this in mind, the volume of PEDOT:PSS is adjusted with deionized water to maintain the required concentration. Glycol is added to the mixture to increase the conductivity as desired. The final step, sodium hydroxide (NaOH) is added to the mixture to allow crosslinking. Various ratios of PEDOT:PSS—Glycol with DA-HA will result in different conductivities. This was tested using a four probe measurement and impedance spectroscopy.

### Fabrication of HMN Patch.

A 3D printed setup on top of the mold allows for a thicker base of the MN patches and attachment of a wire for electrical connections. After pouring the solution, the patches are centrifuged at 7000 rcf for 6 mins which allows the mixture to occupy the needles in the mold. The patches with the wires are allowed to dry inside a nitrogen cabinet overnight.

### EIS

0, 1, 2.5, 5, 10, and 15 wt % PEDOT:PSS thin films were created by spreading the patch mixture over a smooth surface and left to try in a nitrogen desiccator overnight (wt % relative to DAHA). Thin films acted as the WE, Ag/AgCl was used as RE, and Pt as the CE. All electrodes were immersed in 1x PBS as the aqueous medium.

### Porosity Experiment

0, 2.5, and 5 wt % PEDOT:PSS thin films were allowed to swell in water for 10 mins (wt % relative to DAHA). The films were then snap frozen with liquid nitrogen and freeze dried for 48 hours.

### Swelling Experiment

0, 2.5, and 5 wt % PEDOT:PSS HMN patches (wt % relative to DAHA) were cut around the needle array boarder and weighed prior to swelling (wₒ). They were then inserted onto a parafilm-covered 1.4% agarose in 1x PBS gel pad for 10 min. The patches were pressed onto the top of the pad so that the needles puncture through the parafilm and gel, to mimic the stratum corneum of skin. Patches were removed and their final weight was recorded (W_{f}). The HMN patch s e ling was then calculated based on this formula: swelling=(w_{f}−wᵢ)/wᵢ*100.

### Ex Vivo Chronoamperometry Experiment

Porcine ears were obtained and the layer of skin was separated from the cartilage. The skin was then soaked in pH buffer at 4° C. for 12 hours prior to the experiment in order to equilibrate. The skin was removed from the buffer and pat dry with paper towel in order to remove any excess buffer prior to the experiment. The three-electrode system was then mounted on the skin and connected to the potentiostat. The WE used was the 5 wt % PEDOT:PSS HMN patch (wt % relative to DAHA), RE was a Ag/AgCl MN patch, and CE was an Au MN patch.

### In Vitro Chronoamperometry Experiment

1.4% agarose in 1X PBS gel pads were created to reflect the desired pH value. The three-electrode system was then mounted directly on the agarose pad and connected to the potentiostat. The WE used was the 5 wt % PEDOT:PSS HMN patch (wt % relative to DAHA), RE was a Ag/AgCl MN patch, and CE was an Au MN patch.

### pH Step Experiment

5 wt % PEDOT:PSS thin films were used as the WE (wt % relative to DAHA), Ag/AgCl and Pt were used as the RE and CE, respectively. 1x PBS was used as the aqueous medium, and pH was adjusted by adding potassium dihydrogen phosphate (KH2PO4) and NaOH at 30 s intervals. Between each interval, the adjusted PBS solution was allowed to sit for 1 min to equilibrate after stirring prior to performing electrical measurements

### 2.5 References

[3] J. J. Garcia-Guzmán, C. Pérez-Ràfols, M. Cuartero, and G. A. Crespo, “Toward In Vivo Transdermal pH Sensing with a Validated Microneedle Membrane Electrode,” ACS Sens., vol. 6, no. 3, pp. 1129-1137, March 2021, doi: 10.1021/acssensors.0c02397.
[⁵] G. K. Mani et al., “Microneedle pH Sensor: Direct, Label-Free, Real-Time Detection of Cerebrospinal Fluid and Bladder pH,” ACS Appl. Mater. Interfaces, vol. 9, no. 26, pp. 21651-21659, July 2017, doi: 10.1021/acsami.7b04225.
[15] S. Nakata, M. Shiomi, Y. Fujita, T. Aria, S. Akita, and K, Takei, “A wearable pH sensor with high sensitivity based on a flexible charge-coupled device,” Nat Electron, vol. 1, no. 11, pp. 596-603, November 2018, doi: 10.1038/s41928-018-0162-5.
[20] M. Zheng et al., “Osmosis-Powered Hydrogel Microneedles for Microliters of Skin Interstitial Fluid Extraction within Minutes,” Advanced Healthcare Materials, vol. 9, no, 10, p. 1901683, 2020, doi: 10.1002/adhm.201901683.

[21] H. Chang et al., “A Swellable Microneedle Patch to Rapidly Extract Skin Interstitial Fluid for Timely Metabolic Analysis,” Advanced Materials, vol. 29, no. 37, p. 1702243, 2017, doi: 10.1002/adma.201702243.

[33] W. Wang, M. Cui, Z. Song, and X. Luo, “An antifouling electrochemical immunosensor for carcinoembryonic antigen based on hyaluronic acid doped conducting polymer PEDOT,” RSC Advances, vol. 6, no. 91, pp. 88411-88416, 2016, doi: 10.1039/06RA19169J.
[36] E. Lih, S. G. Choi, D. J. Ahn, Y. K. Joung, and D. K. Han, “Optimal conjugation of catechol group onto hyaluronic acid in coronary stent substrate coating for the prevention of restenosis,” J Tissue Eng, vol. 7, p. 2041731416683745, December 2016, doi: 10.1177/2041731416683745.
[37] [symbol] , [symbol] , [symbol] , and [symbol] , “Purification method for hyaluronic acid and/or salts thereof,” WO2011114475A1, Sep. 22, 2011 Accessed: Jul. 26, 2021. [Online]. Available: https://patents.google.com/patent1WO2011114475A1/en
[38]. M. Dervisevic, et al. Transdermal Electrochemical Monitoring of Glucose via High-Density Silicon Microneedle Array Patch. Adv. Funct. Mater. 2009850, 1-10 (2021).
[39]. K. B. Kim, et al. Continuous glucose monitoring using a microneedle array sensor coupled with a wireless signal transmitter. Sensors Actuators, B Chem. 281, 14-21 (2019).
[40]. S. Kim, et al. Electrochemical deposition of dopamine-hyaluronic acid conjugates for anti-biofouling bioelectrodes. J. Mater. Chem. B 5, 4507-4513 (2017).

### Example 3: Supplementary Information

### Methods and Materials—pH Sensing

Chemicals and Materials: Sodium Hyaluronate Hyaluronic Acid (HA) was purchased through Bloomage Biotech (Hybloom™ Low Molecular Weight Sodium Hyaluronate (HA-TLM)). Dopamine hydrochloride (2-(3,4-Dihydroxyphenyl)ethylamine hydrochloride), 1-ethyl-3-(3-dimethylaminopropyl)carbodiimide (EDC) hydrochloride, N-hydroxysuccinimide (NHS), Dialysis tubing cellulose membrane, Monobasic potassium phosphate (KH2PO4) and Potassium hydrogen phthalate were purchased from Sigma-Aldrich. Milli-Q water was used as deionized water in hydrogel synthesis and experiments. PEDOT:PSS (1.3 w.t. %) was obtained from Ossila, and Microneedle molds were procured via Micropoint Inc (Singapore). Porcine skins were obtained from a local super market.

Used herein: 0. 1, 2.5, 5. 10, and 15 wt % PEDOT:PSS (where wt % is relative to DAHA, as used in Examples 2 and 3) is respectively equivalent to 0, 6.4, 16, 32, 64, and 96 vol % PEDOT:PSS (where vol % is relative to total liquid composition, as used in Example 2).

Fabrication of MN-RE and MN-CE: UV epoxy was poured over the MN molds and centrifuged at 7000 rcf for 6 mins. The patches were then cross-linked under UV light. Once removed from the mold, 250 thick Au was evaporated overtop a 2 nm chromium seed layer (intelvac thermal evaporator) to create the CE. Ag/AgCl solution was drop casted over the patch and spread using an air gun to create the RE.

Electrical Characterization: 0, 1, 2.5, 5, 10, and 15 wt % PEDOT:PSS thin films were created by spreading the patch mixture over a smooth surface and left to try in a nitrogen desiccator overnight. For EIS measurement, thin films acted as the WE, Ag/AgCl was used as RE, and Pt as the CE. All electrodes were immersed in 1x PBS as the aqueous medium and a potentiostat (Palmsens4) was used. 4-point-probe measurement was done using Signatone (SP4-40180TRS).

Mechanical Compression Experiment: The mechanical strength testing was performed using the Instron 5548 micro tester, 0, 1, 2.5, 5, 10, and 15 wt % PEDOT:PSS HMN patches were cut around the needle array boarder and mounted on an anvil set to a final height of 100 mm. A compressive load was gradually added with a sled weight of 10 N.

Pore Area Calculation: Raw SEM images were imported into ImageJ and converted to 8-bit binary images. Image threshold was set to 178 and hole outlines were created. Particles were analyzed using a built-in area function, with a size constraint set to a minimum pore area of 5 μm².

Reproducibility Experiment: HMN electrodes were inserted into equilibrated porcine skins with pH 7 solution and chronoamperometric measurements were taken. After the measurements, the HMN electrodes were removed from the skin and allowed to dry overnight in a nitrogen desiccator. This measurement process was repeated over a span of five days.

Stability Experiment: HMN electrodes were inserted into fresh porcine skin and placed in a −20° C. freezer for a set number of days (to minimize skin rot). On the assigned measurement day, the electrode-skin combo was allowed to thaw and HMN electrodes were removed from the skin and dried in a nitrogen desiccator. Once dry, HMN electrodes were re-inserted in the skin and chronoamperometric measurements were taken.

Rigidity Experiment: In order to assess needle integrity, HMN electrodes were imaged before and after a 10 min insertion into porcine skin. The HMN electrodes were then allowed to dry in a nitrogen desiccator overnight. This process was repeated 4 times.

Biocompatibility experiment: The biocompatibility of the three MN electrode system was assessed using an Methylthiazolyldiphenyl-tetrazolium bromide (MTT) assay with mouse fibroblast cells (NIH/3T3). 50,000 cells/well at a total volume of 200 μL were seeded in a 24-well plate. The biocompatibility of the MN-CE and MN-WE was assessed by placing the electrodes directly into the wells, with a 24 hour incubation. The biocompatibility of the HMN-WE was assessed by adding the HMN patch solution (10 μL) into each well for 24 hours. DMEM medium (10 μL) was used as a control. Next, a 5 mg/mL MTT stock solution (20 μL) was added to the wells and incubated away from light for 3 hours. DMSO was used to break up the cells (300 μL) and samples were transferred to a 96-well plate for UV absorbance measurements at 540 nm.

H&E Staining Experiment: HMN patches were applied on the shaved back of a rat for 5 mins. The rat was euthanized after removing the HMN patches, and the skin section with the dermal micropores was cut and washed with 0.9% NaCl solution. The sample was fixed with neutral buffered 10% formalin for 24 hours, then stored in 70% ethanol at −20° C. The embedding, sectioning, and H&E staining were conducted by PhotoMedicine Labs in the Department of Systems Design Engineering at the University of Waterloo.

In-vivo pH Measurement: In-vivo experiments were done following the Guidelines for the Care and Use of Laboratory Animals and the Animal Welfare Act Regulations; all protocols were approved by the University of Waterloo Institutional Animal Care and Use Committee. Male Sprague dawley rats were anesthetized using isoflurane and the hair was removed from their dorsal side using an electric shaver and Nair. The three MN electrode system was mounted onto the dorsal skin and pressed through the epidermal layer. Chronoamperometry measurements (90 s and 180 mins) were carried out at 0.4 V using the Palmsens4 potentiostat, with the HMN-WE, MN-CE, and MN-RE. To validate the three MN electrode system, a commercial pH meter (Orion 9863BN micro pH electrode) was used to measure rat ISF pH. To do so, a subcutaneous incision was made through the rat's dorsal skin and the pH meter was inserted under the skin.

Statistical Analysis: All statistical analysis was conducted using GraphPad Prism 9.0. The statistical difference between groups in the stability test was analyzed using ordinary one-way ANOVA with multiple comparisons follow up t-tests using Tukey's correction. The statistical difference between the readings from our HMN-pH meter and a commercial pH meter was analyzed using two-tailed unpaired t-tests with Welch's correction. All statistical differences were calculated with a 95% confidence interval (P<0.05) and shown in GP style (0.1234 (ns), 0.0332 (*), 0.0021 (**), 0.0002 (***), <0.0001 (****)). The ex-vivo calibration curve and the mechanical strength correlations were calculated by simple linear regression. The pH-step and in-vivo rat pH measurements contain two replicates. The mechanical strength and in-vitro chronoamperometry experiments contain one replicate. The ex-vivo calibration curve contains four replicates. All other experiments contain three replicates. All data is expressed as mean±SEM.

### Example Calculation for HMN Patch Fabrication:

### Mass of DAHA (mg):

m_{DAHA}=(50 mg)(# patches)

Volume of PEDOT:PSS stock solution (μL):

bol_{PEDOT}=CF(desired PEDOT fraction)(mass DAHA)

### Where:

$$CF = 76.92 = \frac{100}{1.3} to account for 1.3 wt \% stock solution$$

Volume of glycol (μL):

vol_{glycol}=(0.1)(vol_{PEDOT:PSS})

Volume DI water (μL):

vol_{DI}=(#patches)(600 μL)−vol_{PEDOT:PSS}−vol_{glycol}

Example calculation: 1 HMN patch with 5 wt % PEDOT:PSS

### Mass DAHA=50 mg

Volume PEDOT:PSS stock=192 μL
Volume glycol=19.2 μL
Volume DI water=388.5 μL

vol_{5% PEDOT:PSS}=(76.92)(0.05)(50 mg)=192.3 μL

vol_{flycol}=(0.1)(192.3 μL)=19.2 μL

vol_{DI}=(1)(600 μL)−192.3 μL−19.2 μL=388.5 μL

### Further Characterization of HMN Patch Fabrication—PH Sensing

A suit of experiments focused on further characterization of the HMN fabrication process was optimized. Since PEDOT:PSS provides conductivity to the HMN-WE, a sufficient amount of PEDOT:PSS should be added to ensure good conductivity, while still maintaining needle integrity for skin penetration. PEDOT:PSS concentrations were taken as a percentage of DA-HA used to make each HMN patch,

The conductivity levels of different PEDOT:PSS concentrations in the patch solution was first assessed. Electrical impedance spectroscopy (EIS) (FIGS. 13a) and 4-point probe measurement (FIG. 13b) were conducted for thin film samples of varying PEDOT:PSS concentrations. EIS showed that impedance decreases as PEDOT:PSS concentration increases, and 4-point probe data compliments this trend, showing a linear increase in conductivity with an increase in PEDOT:PSS concentration. The electrical conductivity measured for the PEDOT:PSS DA-HA mixture fell in the semiconductor range for electroactive polymer composites,[40] having a similar order of magnitude to that of common conducting polymers such as poly(p-phenylene) (10⁻³-10² S cm⁻¹) and polyaniline (10⁻²-10⁰ S cm⁻¹).[41] This data supported that increasing PEDOT:PSS concentrations allows for enhanced HMN-WE conductivity.

Next, mechanical strength testing was performed (FIG. 13c) to determine whether the HMN patches had enough compressive strength to effectively pierce the epidermis. Force vs. displacement plots were obtained showing the load force sustained by a single MN before its point of plastic bending (sharp increase in force vs. displacement). The slope of the force vs. displacement associated with each MN's point of plastic bending showed a decreasing linear relationship with respect to an increasing PEDOT:PSS concentration (FIG. 13c-inset), PEDOT:PSS acted as a plasticizer, providing the HMNs with increased flexibility. Despite the needle flexibility, the force vs. displacement plots showed that the HMNs can withstand a force greater than 0.6 N/needle without fracture, which is greater than the 0.3-0.4 N needle⁻¹ fracture force commonly reported.[42], [20] Since lower flexibility is generally preferable for successful epidermal penetration, HMNs with lower PEDOT:PSS concentrations were considered advantageous.

The effect of introducing PEDOT:PSS on the swelling capability of HMN patches was assessed using an agarose hydrogel model that mimicks the skin environment (FIG. 13d) and through measuring the porosity of hydrogel thin films. HMN patches were applied on the agarose hydrogel for 10 mins, and the patch's weight was measured before and after application. FIG. 13d shows that decreasing PEDOT:PSS concentration increased swelling, thus allowing for a larger ISF volume uptake.

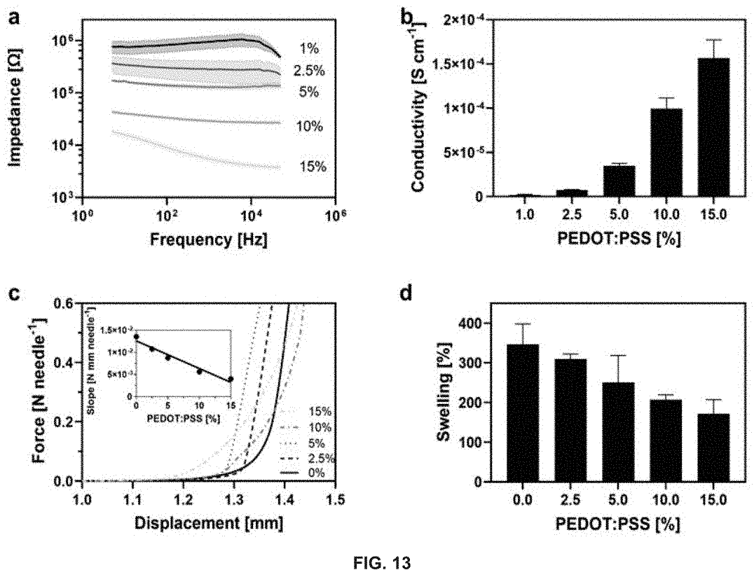

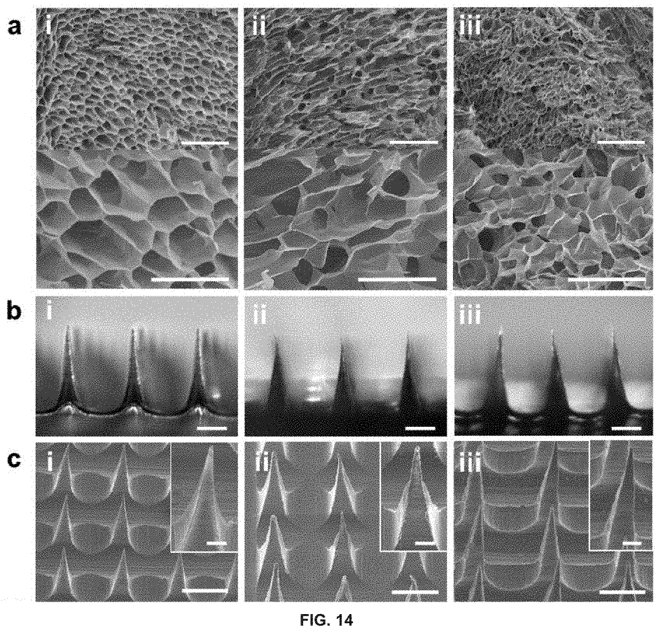

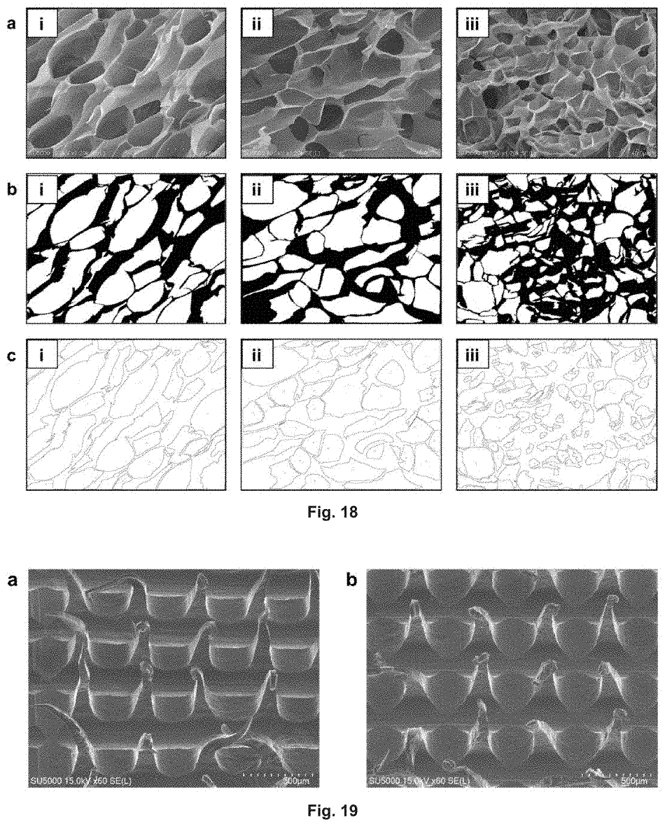

The swelling capability of HMNs depends on their porous structure. To study the porosity, thinfilms were made from 0, 2.5, and 5 wt % PEDOT:PSS patch solutions and were allowed to swell in water for 10 mins. Scanning electron microscopy (SEM) images (FIG. 14a) indicated that higher PEDOT:PSS concentration lead to a more dense and less porous network, with an average pore area of 202, 127, and 47 pmt for the 0, 2.5, and 5 wt % films, respectively (FIG. 18). This data supported the swelling discussed in FIG. 13d, emphasizing that an increased pore size allows for a larger ISF uptake volume.

Optical and scanning electron microscopy (SEM) imaging of HMN patches were performed to ensure that the addition of PEDOT:PSS does not interfere with needle integrity. Optical images of the two dimensional (2D) needle arrays (FIG. 14b) as well as SEM images of three dimensional (3D) needle arrays (FIG. 14c) were analyzed. Increased PEDOT:PSS concentrations resulted in less ridged needles and introduce more flexibility into the patches, which is generally not favorable for skin penetration. In addition, 10 wt % and 15 wt % PEDOT:PSS HMNs did not appear to have enough structural integrity to be used as a HMN-WE, since their increased flexibility causes needles to distort when separated from the PDMS mold (FIG. 19).

According to the above measurements, it was found that the 5 wt % PEDOT:PSS concentration presented a good balance between conductivity and needle integrity. The 5 wt % PEDOT:PSS patches had enough strength to penetrate the epidermis, were able to swell to 250% their original size, presented good conductivity (3.46 E-05 S cm⁻¹), and had a low impedance (140 kΩ).

### Ex-Vivo pH Measurement using a Skin Model—pH Sensing

Having determined a suitable PEDOT:PSS concentration, the pH sensing capability of the HMN patch was investigated.

pH is a measure of H⁺ ions (Equation 1), which can be quantified with the DA-based pH meter. Current is a measure of electron flow through a medium, and the ionic concentration of a solution affects its ability to conduct electricity. As denoted by Equation 2, conductivity (σ) is directly proportional to current (I). In this equation, A, V, and L represent cross-sectional area, applied voltage, and length of the electrode, respectively. At lower pH, due to the increase of H⁺ ions in the system, DA is reduced to its catechol form, bearing a neutral charge. Comparably at higher pH, DA is oxidized to its quinone form, bearing a negative charge. The negative charge build-up on the electrode due to the quinone not only increases conductivity, but increases the capacitance of the electric double layer. Combining the capacitance with Ohm's Law, it can be seen that current (I) should increase as charge build-up (Q) increases (Equation 3). Equation 3 shows this relationship, where C is the capacitance and R is the resistance. Therefore, it was reasonably deduced that the measured current should be higher in solutions with higher pH, due to the oxidation of DA.

pH=−log[H⁺] (1)

σ=IL/AV (2)

I=Q/CR (3)

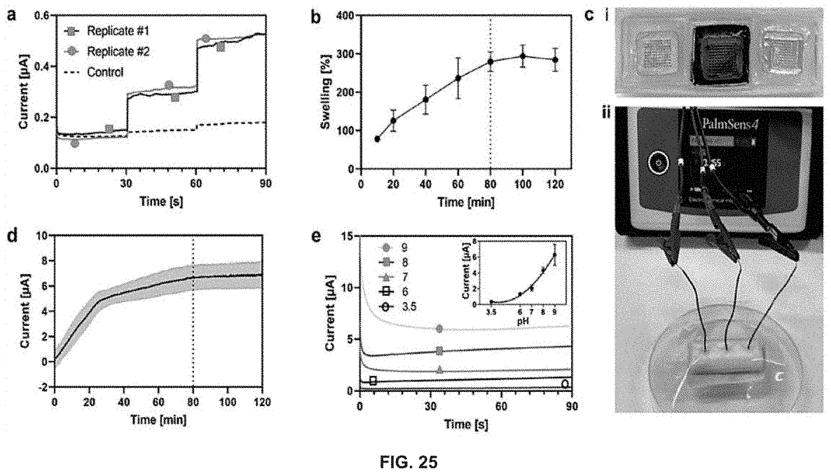

To investigate this hypothesis, a pH step increase was completed (FIG. 25a). 5 wt % PEDOT:PSS thin films were used as the WE and were immersed in a pH-controlled buffer solution. It was observed that the current increased as pH increased, with an approximate 2-fold increase between pH 6, 7, and 8. On the other hand, a negligible increase in current was seen for the control sample with no pH increase.

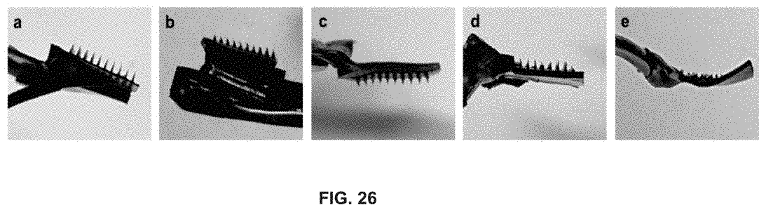

Prior to examining the HMN patch for pH sensing, the time required for the patch to reach its maximum swelling was determined. HMN patches were applied into porcine skin for different durations, and it was determined that 80 mins allowed for the HMN to reach its maximum swelling in a skin environment (FIG. 25b). It was noted that the swelling behavior of HMN patch in skin was different from the agarose hydrogel model discussed in FIG. 13d, due to the lower water content of skin compared to the agarose hydrogel. HMN rigidity was also assessed by performing multiple insertions into porcine skin. FIG. 26 shows that the needles remained intact and preserved their integrity up until the third insertion, demonstrating the rigidity of the needles for pH measurement.

Next, the current response of the HMN-WE was assessed in ex-vivo conditions by mounting the three MN electrode system (FIG. 25ci) on porcine skin and connecting it to the potentiostat (FIG. 25cii). The long-term signal response time for the HMNs was determined by assessing the effect of swelling saturation on the measured current (FIG. 25d). An 80 min current response time was determined, which correlated to the 80 mins it took for the HMNs to reach their maximum swelling. The increase in current during the swelling stage can be attributed to the increase of pore sizes and fluid interaction with more active surfaces inside the HMNs. The HMN-pH meter was able to report a stable current for the next 40 mins after reaching a plateau.

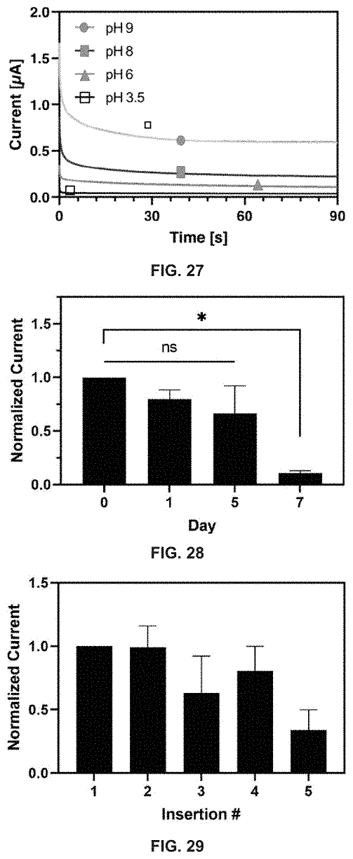

The HMN-WE's ability to detect pH changes in-vitro on pH-controlled agarose hydrogels (FIG. 27) and ex-vivo on porcine skins (FIG. 25e) was then assessed, and a clear current increase was observed with increasing pH levels. Chronoamperometry measurements were carried out on skins that were equilibrated to a clinically relevant pH range of 3.5, 6, 7, 8, and 9 (FIG. 25e)^{[33]} and a calibration curve was created (FIG. 25e-inset). A duration of 90 s was chosen to demonstrate the proof-of-concept for real-time and single-point measurements. The measured current increases as pH increases, indicating that our HMN-pH meter is capable of on-needle detection and differentiation of varying pH levels without any post-processing required.

To take this further, the HMN-WE's signal stability was assessed over seven days by comparing the electrochemical signal obtained to that of a fresh HMN patch. It was observed that the HMN's signal change over a five day storage period remained statistically insignificant to the signal measured on day zero (FIG. 28), It was observed that the HMN lost about 95% of its signal by the seventh day, suggesting that it may not be suitable for periods extending five days. Possible reasoning for this signal loss was due to saturation of DA-HA's catechol functional group as a result of the porcine skin gaining acidity from slight decay. ^{[47]}

Also evaluated was the HMN-WE's signal reproducibility over multiple insertions in porcine skin (FIG. 29). It was observed that 20% and 66% of the initial signal was lost by the fourth and fifth insertions, respectively.

### In-vivo PH Measurement using a Rat Model—PH Sensing

Prior to introducing the electrodes into an in-vivo system, biocompatibility experiments were conducted using a methylthiazolyldiphenyl-tetrazolium bromide (MTT) assay, showing a retention of 99, 88, and 88% cell viability for the HMN-WE, MN-CE, and MN-RE, respectively. With the biocompatibility of the system investigated, the three MN electrode system was introduced into a rat model for real-time in-vivo pH sensing.

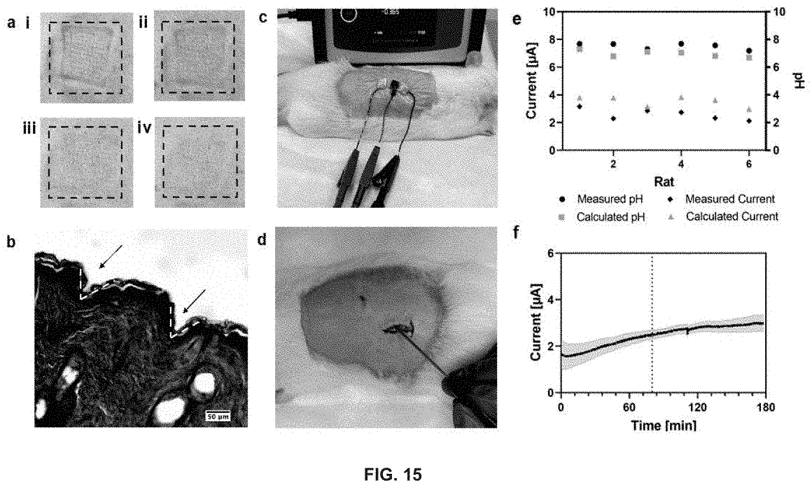

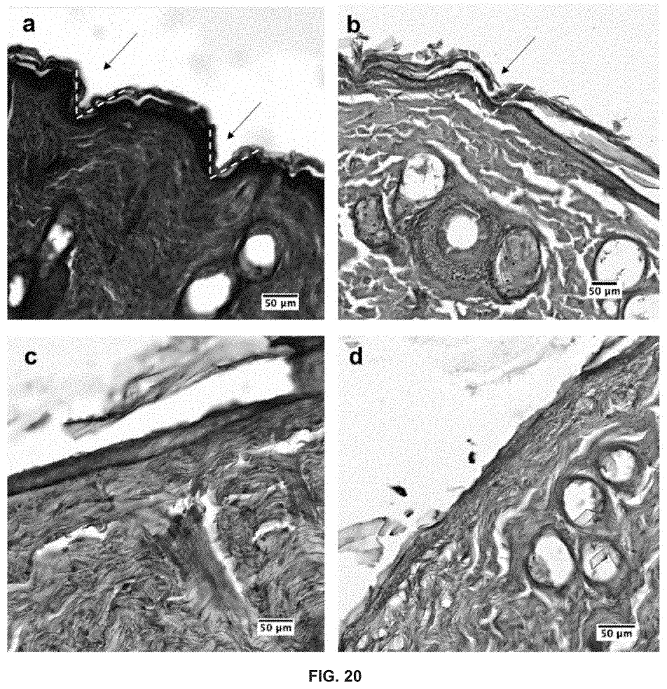

The effectiveness of the HMN-WE penetration and healing time were first assessed. Clear MN_insertion traces were observed (FIG. 15a), and healed after 10 mins, while the MN-CE and MNRE traces healed after 10 mins and 5 mins, respectively. Also examined were the healing time and inflammatory response in tissues surrounding the HMN penetration sites through haematoxylin and eosin (H&E) staining of live rat skin. FIG. 15b showed that the HMN penetrated the skin at a depth of 102±6 μm (n=5, mean±SEM) with no visible signs of inflammation around the micropore compared to the control skin (FIG. 20). As shown in FIG. 20, the skin completely healed at the cellular level 30 mins upon the patch removal, This data indicated that the HMN-based sensor was a minimally invasive technique that did not appear to cause any inflammation or long-term damage.

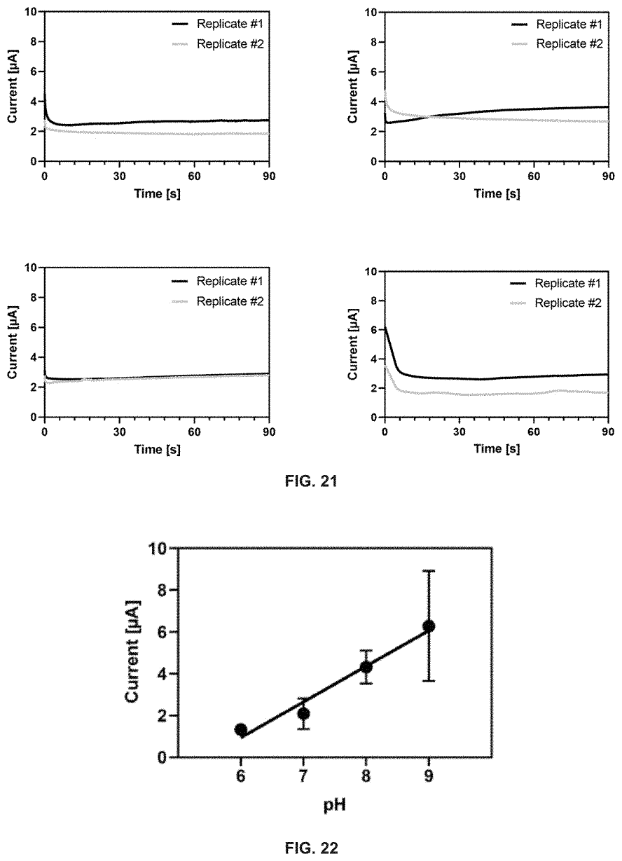

Next, the ability of the HMN-pH meter to measure in-vivo pH was assessed. The three MN electrode system was inserted on the dorsal side of the rat and connected to the potentiostat as shown in FIG. 15c. The current response was measured in six rats by chronoamperometry (FIG. 21) and closely matched the current response of a ex-vivo model (for example, see above ‘Ex-vivo pH measurement using a skin model’).

Two methods were employed to validate the in-vivo sensing model: validation by the ex-vivo model and validation against a commercial pH probe. Firstly, the ex-vivo model was used to calculate the pH of the ISF from the measured current, using the linear section of the calibration curve (FIG. 22). The calculated pH was then compared against readings from a commercial pH probe that was inserted into the ISF under the rat's subcutaneous tissue (FIG. 15d). Secondly, the pH measured from the commercial probe was interpolated into the ex-vivo model to calculate the theoretical current, FIG. 15e shows the difference between actual and calculated pH and current values for all six rats tested. An average difference of 7.4% was calculated for pH measurements, and all measurements from the HMN-pH meter were found to be statistically insignificant from those of the commercial pH meter. Average calculated values and standard errors are reported in Table S1 and P-values from significance testing are reported in Table S2.

To study the HMN-pH meter's prolonged monitoring, the HMN-pH meter was for pH measurement in a sedated rat for 3 hours (FIG. 15f). After approximately 80 min of device administration, the measured current reached its maximum level and stayed at this level for the next 100 mins.

TABLE S1
Measured current and pH values for in-vivo model.
Data reported as mean ± SEM with n =
2 replicates for current and n = 3 replicates for pH.

| Rat # | Measured Current (uA) | Measured pH |
|---|---|---|
| 1 | 3.16 ± 0.487 | 7.68 ± 0.029 |
| 2 | 2.74 ± 0.423 | 7.69 ± 0.012 |
| 3 | 2.33 ± 0.623 | 7.57 ± 0.020 |
| 4 | 2.12 ± 1.263 | 7.19 ± 0.007 |
| 5 | 2.30 ± 0.454 | 7.67 ± 0.009 |
| 6 | 2.85 ± 0.053 | 7.30 ± 0.1135 |

TABLE S2
Significance testing between results obtained from
the HMN-pH meter and the commercial pH meter as per
two-tailed unpaired t-tests with Welch's correction.

| Rat # | Current | pH |
|---|---|---|
| 1 | ns (P = 0.4075) | ns (P = 0.4066) |
| 2 | ns (P = 0.1336) | ns (P = 0.1339) |
| 3 | ns (P = 0.0882) | ns (P = 0.0856) |
| 4 | ns (P = 0.2340) | ns (P = 0.2365) |
| 5 | ns (P = 0.2835) | ns (P = 0.2848) |
| 6 | ns (P = 0.6236) | ns (P = 0.6237) |

### Methods and Materials—Glucose Sensing

Materials. Hyaluronic add (HA, MW 300 KDA) was purchased from Bloomage Co,, Ltd (China). 1x PBS, Dimethyl sulfoxide (DMSO, 25-950-CQC), was purchased from Corning, USA. Dopamine hydrochloride (DA), N-(3 Dimethylaminopropyl)-N′-ethylcarbodiimide (EDC), N-Hydroxysuccinimide (NHS), Sodium hexachloroplatinate(IV) hexahydrate, ascorbic acid (AA), uric acid (UA), magnesium chloride (MgCl2), potassium chloride (KCl), ethylene glycol (EG), silver nitrate (AgNO3), UV curable resin (55 cp), silver/silver chloride (Ag/AgCl) (60/40) paste for screen printing, agarose type I-A (A0169), Methylthiazolyldiphenyl-tetrazolium bromide (MTT), and dialysis tubing cellulose membrane (MWCO=14,000) were purchased from Sigma Aldrich (Canada). PEDOT:PSS (PH 1000—Heraeus Clevios™) was purchased from Ossila. Conductive silver epoxy was purchased from MG Chemicals. Tegaderm tapes was acquired from 3M. The porcine ear skin was obtained from a local supermarket,

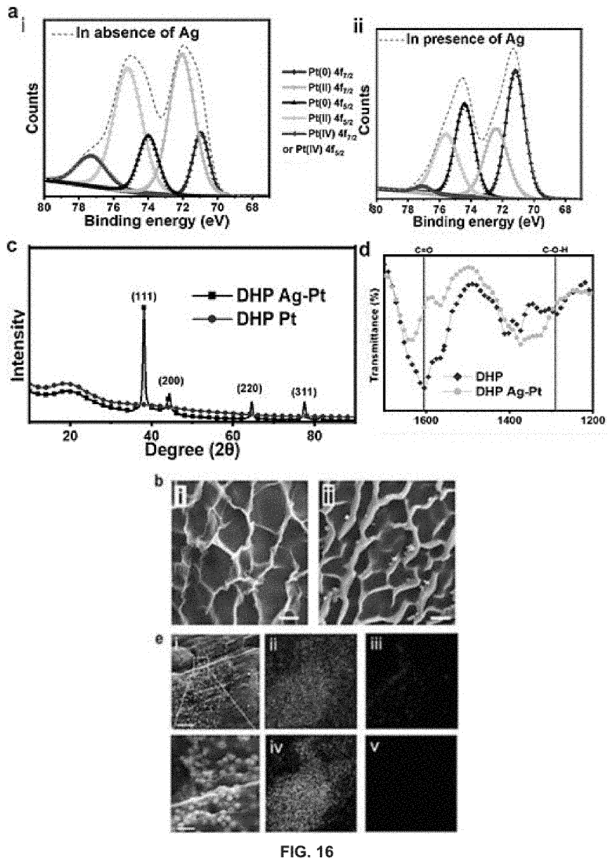

Hydrogel composite characterization using different spectroscopy techniques. The surface morphology and porosity of hydrogel thin films (FIGS. 16b) were characterized by scanning electron microscopy (SEM, Zeiss Ultra Plus). Before acquiring SEM images, the hydrogel thin films were kept inside the DI water overnight for maximum swelling. Next, swelled films were transferred to liquid nitrogen. Then, the frozen hydrogels were freeze-dried. The hydrogel thin films without undergoing swelling (FIGS. 16e) were also investigated by SEM and their elemental composition were verified using energy dispersive X-ray spectroscopy (EDX) (Oxford Instrument). The formation of nanoparticles and chemical composition of hydrogels were confirmed by X-ray photoelectron spectroscopy (KPS) (Thermo VG MicroLab 350), X-Ray diffraction (XRD) (D8 Discover, Bruker) technique, and Fourier transform infrared (FTIR) spectroscopy (Tensor 27 FTIR, Bruker).

Porosity characterization via image analysis. The SEM images were analyzed by the lmageJ software. The percentage of porosity was calculated by measuring the number of dark pixels in hollow areas divided by the whole picture pixels.

Electrical characterization. For Electrochemical Impedance Spectroscopy (EIS) measurement of thin films, a conventional three-electrode electrochemical cell and a potentiostat (Palmsens4) were used, After cutting thin films to uniform sizes of 5 mm×15 mm, WE clamp was attached to the thin films. The WE thin film kept inside an electrochemical cell with 20 mL 1x PBS buffer along with a commercial Ag/AgCl (3M NaCl) reference electrode (MF-2052, BASI) and a commercial Pt counter electrode. The measurement was done from 10000 to 10 Hz at room temperature. 4-point-probe measurement was done using Signatone (SP4-40180TRS).

Electrochemical measurement for glucose detection. A conventional three-electrode electrochemical cell and a potentiostat (Palmsens4) were used for the cyclic voltammetry (CV) and chronoamperometry measurement of the films in 1x PBS buffer solution containing different concentrations of glucose, AA, AU, KCl, MgCl2. Each film (5 mm×15 mm) was used as the WE along with a commercial Ag/AgCl reference electrode and a commercial Pt counter electrode. The CV scanning was done at a scan rate of 20 mV/S and a potential range of −0.6 to 0.8 V. For chronoamperometry measurement the potential was set to 0.4 V and measurement was performed for 40 s. For measuring the response of glucose step increase, a stock of 1 M glucose was prepared, and it was added to make 2.5, 7.5, 12.5, 22.5 mM glucose concentrations. Upon adding the reagent (glucose or interferences), the solution was stirred for 20 s to make a homogenous solution, then the stirring was stopped and the measurement was performed. Measurements were repeated at least three times.

To test the HMN-CGM in-vitro and ex-vivo, the HMN-WE along with the MN-RE and MN-CE were penetrated through the agarose hydrogel or porcine skin model loaded with different concentrations of glucose (0-35 mM). The CV or chronoamperometry measurements was carried out as explained above. Measurements for each concentration were repeated at least three times.

### Long Chronoamperometry Measurement using porcine Skin Model.

The porcine ear skins were incubated overnight in 1XPBS with different concentration of glucose (0, 5, 10, and 20 mM). The skins were then dried using tissue papers to remove the moisture from the surface, HMN-WE, MN-CE and MN-RE were then inserted into the porcine skin and their wires were connected to the Potentiostat (CHI 1040C). For chronoamperometry measurement the potential was set to 0.4 V and the measurements were done 3 hr continuously. Measurements for each concentration were repeated at least three times.

### Swelling Experiment using Agarose Skin Mimicking Model.

Agarose hydrogel was prepared by a mixture of 140 mg agarose and 10 mL DI water (1.4%). To make the skin mimicking model, the top surface of agarose gel was covered by a thin layer of parafilm. The HMN patches were inserted through agarose hydrogel by fingertip and kept for 10 min. The HMN patches weight were measured before (W0) and after (Wt) insertion by a digital scale. The swelling ratios were then measured using the below equation:

$$swelling ratio = \frac{{W}_{t} - {W}_{0}}{{W}_{0}} \times 100 \%$$

Long swelling experiment using porcine skin model. The HMN-WE patches were inserted into the fresh porcine ear skin. The patches were held into the skin using transparent Tegaderm tapes. After absorbing the ISF for certain durations (10, 20, 40, 60, 80, 100, and 120 min), the patches (3 replicates for each timepoint) were peeled off from the skin. Then, they were weighted on the scale and the swelling ratio for each patch was calculated based on the above equation.

EIS measurements in porcine skin model The HMN-WE along with MN-RE and MN-CE were inserted into the fresh porcine ear skin for different durations (0, 20, 40, 60, 80, 100, and 120 mins) and the EIS measurements were performed. The patches were held into the skin using transparent Tegaderm tapes. The wires of the 3-electrode MN patches were connected to the potentiostat wires. The DC and AC voltages were set to 5 and 10 mV, respectively and the frequency was changed from 10000 to 10 Hz.

Mechanical test and skin penetration efficiency. The mechanical strength of 5 different HMN arrays were tested using Instron 5548 micro tester. The microtester device was equipped with a 500 N compression cell. The HMN arrays were placed vertically on a compression platen. The distance between two platens was set to 1.5 mm. At a speed of 0.5 mm/min, a compressive force was applied. The compression threshold was set to 60 N. The load and displacement were recorded every 100 ms to derive the load-displacement curve.

Stability characterization. The HMN-WEs were inserted into the 1XPBS equilibrated porcine skin and were kept in the skin for 7 or 14 days. After 7 or 14 days, the patches were removed from the skin and kept inside the nitrogen desiccator for 24 hr for reaching the complete dryness. The patches were then inserted into a dried piece of porcine skin that was previously incubated with 20 mM glucose in 1XPBS. The chronoamperometry measurement was done at 0.4 V for 40 s.

Dynamic mechanical deformation tests. In order to examine the stability of the HMN-CGM signal measurement under real skin movement, a dynamic mechanical deformation test was performed. The 3-electrode MN arrays were inserted into the porcine skin containing 20 mM glucose and the skin was bent or twisted for 100 complete cycles as shown in FIG. 32. Chronoamperometry measurement was measured at 0.4 V to observe the stability of the HMN-CGM signal measurement during these mechanical deformations.

In-vitro cytotoxicity assay and evaluation of biocompatibility. The biocompatibility of three MN electrodes was investigated using mouse fibroblast cells (NIH-3T3). The cells were prepared in a 24-well plate where in each well, 50,000 cells were seeded with a total volume of 100 μL. Then, wells were exposed to 10 μL of sample solution of HMN-WE for 24 hours. The 5 mL sample solution contains 100 mg DA-HA, 380 μL PEDOT:PSS, 38 μL EG, 40 μL from 1M AgNO stock solution and 24 μL from 1M Na₂PtCl₆ stock solution. 10 μL of Dulbecco's Modified Eagle Medium (DMEM) was used as control. Next, all wells were treated by 10 μL of the 5 mg/ml stock solution of Methylthiazolyldiphenyl-tetrazolium bromide (MTT). Next, the 96-well plate was incubated and kept away from light for 3 hr. To break up cells and release the formazan crystals, 150 μL of DMSO was gently added to treated wells. The absorbance of the samples was then measured using a spectrophotometer at 540 nm. The biocompatibility of MN-RE and MN-CE was tested by directly placing the MN electrodes in the wells.

In-vitro Ag and Pt release. The amount of Ag and Pt released from the composite hydrogel was analyzed via inductively coupled plasma optical emission spectrometer (ICP-OES: Thermo iCAP 6500). HMN-WE was inserted on an agarose gel (1.4% w/w) covered with a parafilm layer to mimic skin environment and kept on for 24 hr. The agarose gel was then transferred to a glass vial with 10 mL 1XPBS buffer. The solution was stirred and heated for 30 min at 80° C. to release the Pt and Ag NPs in solution. The remaining agarose was then removed. Next, the calibration was made using Ag and Pt ion concentrations and the solution was analyzed for the presence of Ag and Pt.

In-vivo glucose measurement. In vivo experiments were done following the Guidelines for the Care and Use of Laboratory Animals and the Animal Welfare Act Regulations; all protocols were approved by the University of Waterloo Institutional Animal Care and Use Committee. The streptozotocin (STZ)-induced diabetic rat (T1D) has been used for testing the capability of HMN-CGM device in the animal model. Male Sprague Dawley rats (Charles River,100-150 gr) were injected with STZ (65 mg/kg i.p.), resulting into degradation of the rat's pancreatic beta-cells insulin secretion activity. For 7 days, the blood glucose level of STZ injected rats was measured every day by a conventional glucose meter (Contour® Next ONE meter, Ascensia, Inc., USA). Rats with high blood glucose level (>16 milli) were selected for this study. On the days of experiment, the rats were fasted for 5 hr before starting the measurement. Next, rats were given i.p. injection of 87 mg ketamine/kg and 13 mg xylazine/kg of their weight. After anesthetizing was completed, theft backs were shaved using a shaving machine and application of hair removal cream. Next, insulin (4-5 unit) was injected to the rats subcutaneously and blood glucose levels were measured by the glucose meter every 5 min. HMN-WE, MN-CE, and MN-RE were then attached on a Tegaderm transparent tape (needles faces toward up). The tape with MN electrodes were then applied into the rat's back and the needles were pushed against skin. The chronoamperometry measurement at 0.4 V was then started to measure the ISF glucose level. All measurements were done when blood glucose level decreased to certain ranges (5 ranges in total): T0: 25-35 mM, T1: 15-25 mM, T2: 10-15 mM, T3: 5-10 mM, T4: 3-5 mM.

For prolonged glucose measurement in the healthy rat, an isoflurane vaporizer was used. First, the rat was anesthetized by setting the isoflurane vaporizer rate to 5% for 1 min. Then, the flow rate of oxygen and the isoflurane vaporizer was set to 0.7 L/min and 2.5%, respectively, for the next 3 hr. The MN electrodes were applied in the same manner as described above and the chronoamperometry measurement was recorded at 0.4 V for 3 hr. All the measurements were repeated three times.

Statistical Analysis. Statistical analysis was performed by OriginPro 2021. Values were reported as mean±standard deviation (S.D.). One-way analysis of variance (ANOVA) and student's t-test were used to evaluate the statistical significance. The statistics were considered significant when P<0.05 or less. To estimate the LOD of the HMN-CGM device for glucose measurements, we first measured the electrochemical signal intensity from the blank sample and calculated the mean current plus three times of standard deviation. The LOD was then calculated by interpolating this value into the corresponding calibration curve.^{[48]}

### Characterizing the Formation of Pt NPs in DHP Composite Hydrogel—Glucose Sensing

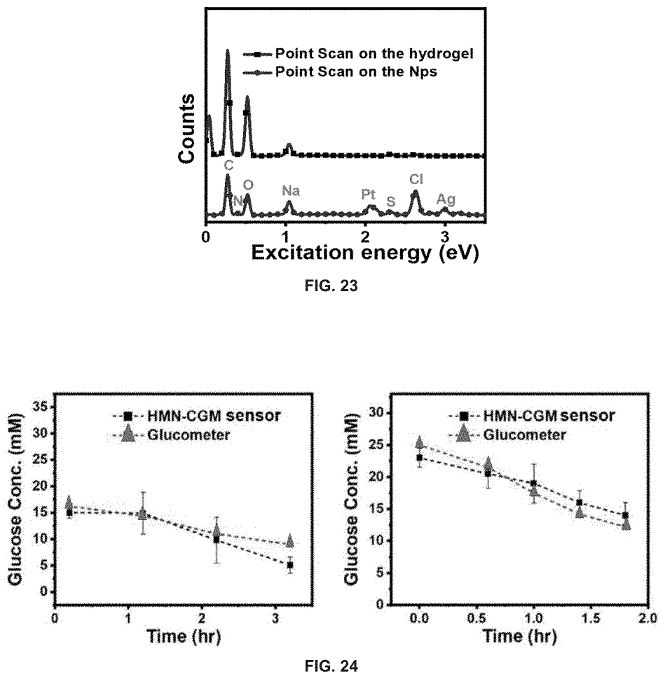

The formation of Pt NPs in a DHP network was studied. The NPs were embedded inside the hydrogel network by in situ reduction of Pt and Ag ions using redox property of catechol moieties of DA.[36] The redox property of catechol was high enough to induce a chemical reduction of Ag ions but not enough for complete conversion of Pt ions to Pt NPs, as the reduction of Pt ions to NPs involves multiple electron transfers.[36-38] However, the partially reduced Pt ions can be fully reduced in the presence of Ag NPs via galvanic replacement to form Ag-Pt hybrid nanostructures.[37,38] The role of catechol and Ag NPs in full reduction of Pt ions and formation of NPs was studied using different spectroscopy techniques (FIG. 16). FIG. 16a shows the X-ray photoelectron spectroscopy (XPS) of Pt 4f orbital region of dried DA-HA hydrogel loaded with only Pt or Ag—Pt ions. The XPS spectrum in Pt 4f orbital region of the samples without Ag revealed two peaks at 72.2 and 75.5 eV (green and cyan) related to the formation of partially reduced Pt (PtO), which was notably higher in value compared to peaks at 71.1, 74.1 and 77.3 eV (red, blue, and pink) that are related to the formation of fully reduced Pt (Pt metal and PtO₂) (FIG. 16ai). Whereas the XPS spectrum of the samples in the presence of Ag showed a notable increase in the concentration of PtO₂ due to the complete reduction of Pt ions to NPs (FIG. 16a-ii). The percentage of fully reduced Pt was calculated to be 61% and 29% in the presence and absence of Ag, respectively, demonstrating that the presence of Ag increases the reduction of Pt ions to the metallic nanostructures. Freeze dried DHP samples were also prepared and imaged by scanning electron microscopy (SEM) (FIG. 16b). No signs of NP formation were found in the samples without Ag metal ions (FIG. 16b-i) while the solid metallic NPs were visible in the sample that contained both Ag and Pt metal ions (FIG. 16b-ii), supporting the XPS measurement results. These samples were further analyzed using XRay diffraction (XRD) technique to confirm the formation of NPs in the composite hydrogel (FIG. 16c). The freeze dried hydrogel in the absence of Ag only showed a broad diffraction peak at 2θ=19.70°, corresponding to amorphous polymers,[39] However, the observed diffraction peaks at 38°, 44°, 64°, and 77° correspond to the (111), (200), (220), and (311) diffractions of metallic particles indicated the presence of the NPs in the sample that contained both Ag and Pt metal ions. Fourier transform infrared (FTIR) spectroscopy was also performed to characterize the chemical composition of the hydrogels before and after addition of metal ions (FIG. 16d). In the DHP spectrum, the peak at 1290 cm⁻¹ which was attributed to C—O—H stretching vibration of catechol group decreased in presence of Ag and Pt ions, suggesting that the oxidation of the catechol moiety facilitated the reduction of metal ions into solid NPs,[39] Moreover, the peaks at 1606 cm⁻¹ which is attributed to C═O of phenol group is shifted to 1565 cm⁻¹ due to the metal—catechol coordination bonding, indicating that the formed NPs were bound tightly to the catechol groups.[39] To further determine the identity of the elemental content and their locations on the sample, energy dispersive X-ray (EDX) mapping analysis was carried out (FIG. 16e and FIG. 23).

The elemental mapping images indicated the presence of both Pt and Ag atoms on the same sites of formed NPs. The results further indicated that the NPs were selectively and uniformly distributed in the hydrogel network. In addition, sulfur and nitrogen elements were visible due to their presence in PEDOT:PSS and DA-HA, respectively. The release of Ag and Pt from HMNs was also analyzed by inductively coupled plasma optical emission spectrometer (ICP-OES). No significant release of Ag and Pt was observed, indicating that the NPs stably coordinated with the catechol groups of the hydrogel composite, which is favorable for minimizing the toxicity effect on the tissues.

### HMN Characterization and Ex-Vivo Glucose Sensing using HMN-CGM

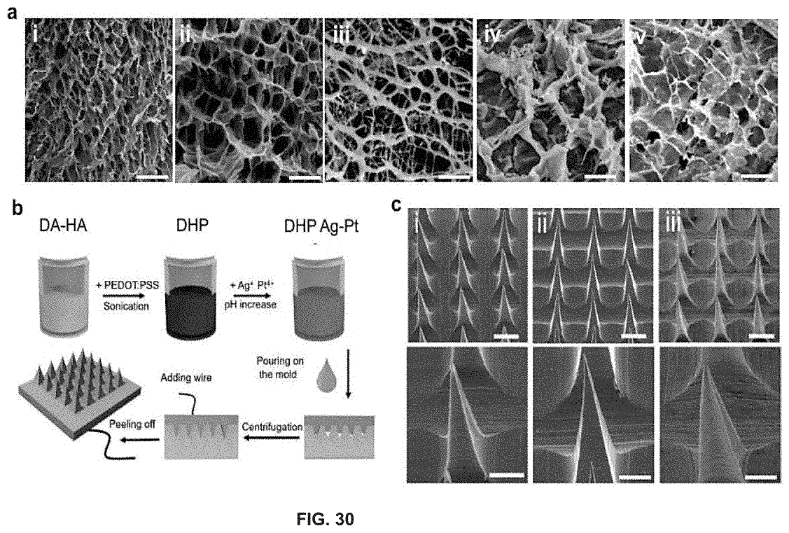

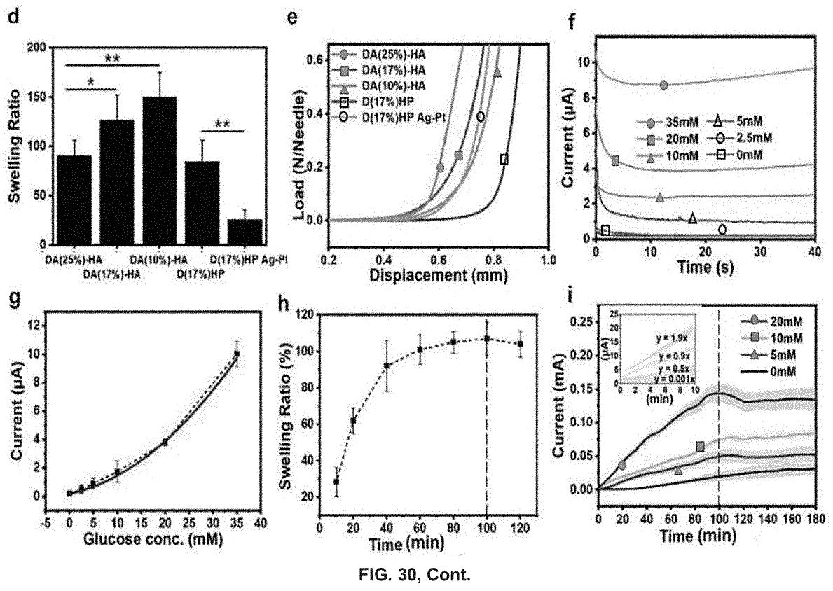

HMN patch fabrication for ISF extraction and skin penetration was characterized. A characteristic of HMN arrays that allows for increased ISF extraction is their porous structure, Hydrogels with three different rates of DA conjugation were synthesized and their porosity was studied. The SEM images of the porous structure of these samples, namely 25, 17 and 10% DA-HA have been shown in FIG. 30 a-i-iii. The 25% conjugation sample showed a more dense and less porous network due to its higher crosslinking degree compared to other samples. Furthermore the pore size of hydrogels reduced but the porosity remained after addition of PEDOT:PSS and NPs (FIG. 30a-iv and 30a-v). Then fabricated was HMN patches using DA-HA along with PEDOT:PSS and NPs (FIG. 301a). It was found that a higher DA conjugation (25%) rate may hinder formation of sharp needles while a low DA conjugation (10%) may impede mechanically strong MNs. A conjugation rate of 17% was found suitable for formation of needles with good integrity and ideal for skin penetration. The tips of HMN fabricated with 17% DA were sharp with 800 μm in height and conical in shape and the addition PEDOT:PSS and NPs did not change the needle integrity and sharpness (FIG. 30c). The swelling capability of HMN arrays fabricated with different components was also examined using agarose parafilm skin model (FIG. 30d). After insertion, HMN arrays were kept on the agarose hydrogel for 10 min and the swelling ratio was measured based on the weight of patch before and after insertion. 10% DA-HA showed a higher swelling ratio compared to 25% and 17% due to less crosslinking degree (FIG. 30a). Upon addition of the NPs, the patch underwent a lower swelling ratio. The lower swelling property of DHP Ag—Pt could be attributed to the presence of NPs that reduced the porosity of the hydrogels. ^{[43}] The ability of HMN arrays with different compositions for skin penetration was also tested by compression test using the dynamic mechanical analyzer (DMA) technique (FIG. 30e). Mechanical compression testing of all tested MN patch demonstrated a mechanical strength of more than 0.62 N/needle that is ideal for successful insertion into skin.^{[3,44]} The addition of PEDOT:PSS increased the slope of the force—displacement rate demonstrating an increase in the flexibility of the HMN patch (gray line) which is not favorable for skin penetration. However, introducing Pt and Ag NPs enhanced the rigidity and thus improved the robustness of the DHP Ag—Pt HMN patch. Then fabricated was the HMN-WE using DHP Ag—Pt composite hydrogel and the glucose sensing in an integrated three MN electrode system, called HMN-CGM device, was investigated. The MN electrodes consisted of an HMN-WE, a solid epoxy MN patch coated with Ag/AgCl screen printing ink as the RE (MN-RE), and an Au coated epoxy MN as the CE (MN-CE).

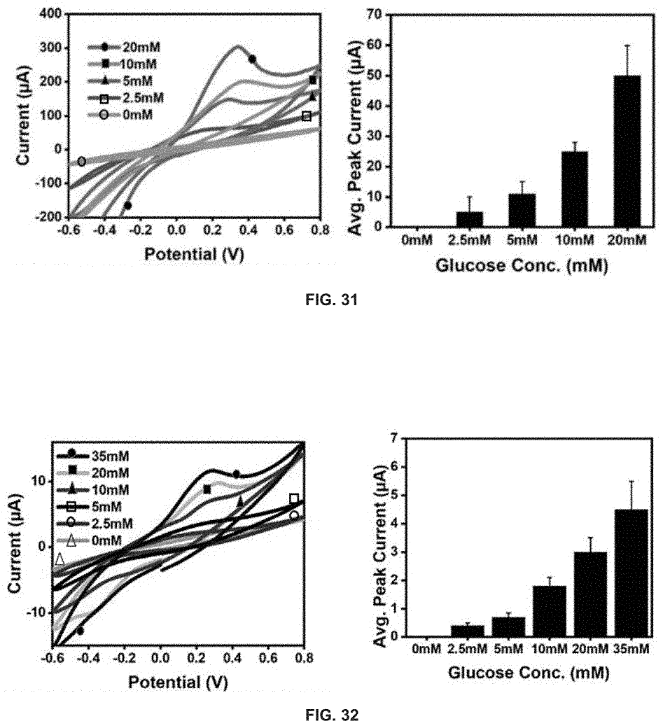

The three MN electrodes were applied on agarose hydrogel (FIG. 31) or porcine ear skin (FIG. 32) containing various glucose concentrations and CV scanning was performed. Similar to the experiments in solution (FIG. 16e), CV scanning resulted in redox peak at 0.2-0.3 V which demonstrated the capability of the sensor for glucose measurement. Then was examined the HMN-CGM for glucose measurement by chronoamperometry measurement as it was suitable for continuous monitoring over the time (FIG. 30f). Increase in current was observed by increasing the glucose concentrations. FIG. 30g shows the calibration curve of current against glucose concentrations extracted at t=40 s. The HMN-CGM device achieved a limit of detection of 0.9 mM for glucose measurement which covered the hypoglycemic range. This was considered an important feature as most of the current CGM devices fail to accurately measure the low level of glucose.^{[45]}

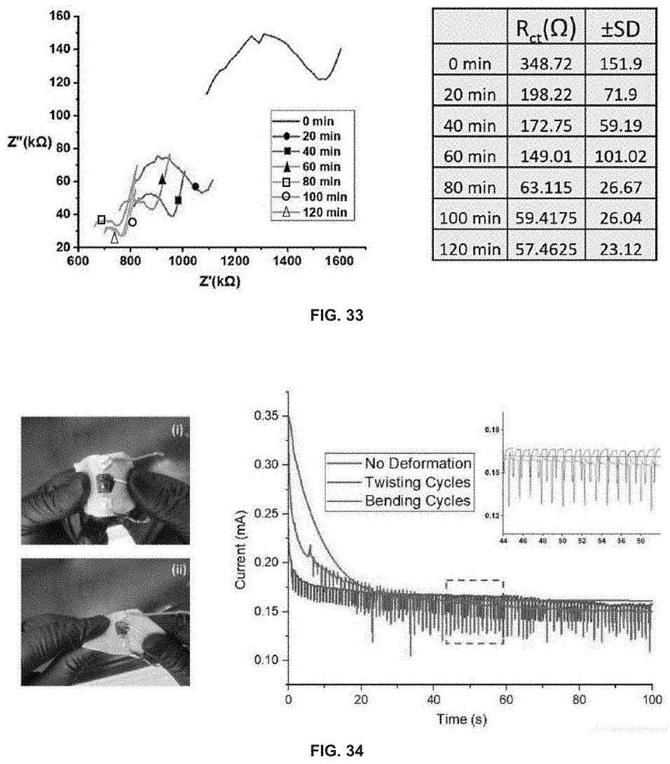

To examine the capability of the HMN-CGM assay for continuous measurement, first determined was the time that it takes for the HMN-WE to reach to its maximum swelling. HMN-WE patch was applied into the porcine skin for different durations and it was observed that approximately 100 min of the patch administration is needed to reach the maximum swelling (FIG. 30h). Then studied was the prolonged measurement of different concentrations of glucose for a course of 180 mins (FIG. 30i). In the first 100 min and during the HMN-WE swelling, the current increased for all concentrations and the HMN-CGM device was able to report a stable current for the next 80 min after reaching to a plateau. The increase in the current may be attributed to decrease in the charge transfer resistance during the swelling stage (FIG. 33). This hypothesis was investigated by by performing EIS measurements where the HMN-WEs were inserted into the porcine skin for different durations (0, 20, 40, 60, 80, 100, 120 mins). It was observed that by increasing the swelling, the charge transfer resistance decreased and the capacitance increased. It was noted that the increase in the current over the time in the first 100 min (the graph slope) was proportional to the glucose level (FIG. 30i-inset) and may be potentially used for glucose measurement. Also examined was the stability of the HMN-WE and found that HMN-WE could be stored in porcine skin for 14 days and remained stable. To assess stability of the HMN-CGM signal measurement under real skin movement and mechanical stresses, a dynamic mechanical deformation test was performed. The performance of the three MN electrode system was evaluated using the porcine skin loaded with 20 mM glucose during deformation of 100 cycles of bending and twisting (FIG. 34). It was observed that the deformations had a negligible effect on the average of electrochemical signals in comparison with a stable skin.

Prior to the in-vivo experiment, the biocompatibility was examined of the components of the three MN electrodes in NIH-3T3 fibroblast cells using MTT assay. Results showed that cell viability was not significantly influenced in the presence of all three MN electrodes, suggesting that the HMN-CGM components were biocompatible, where % cell viability was about ≥80%.

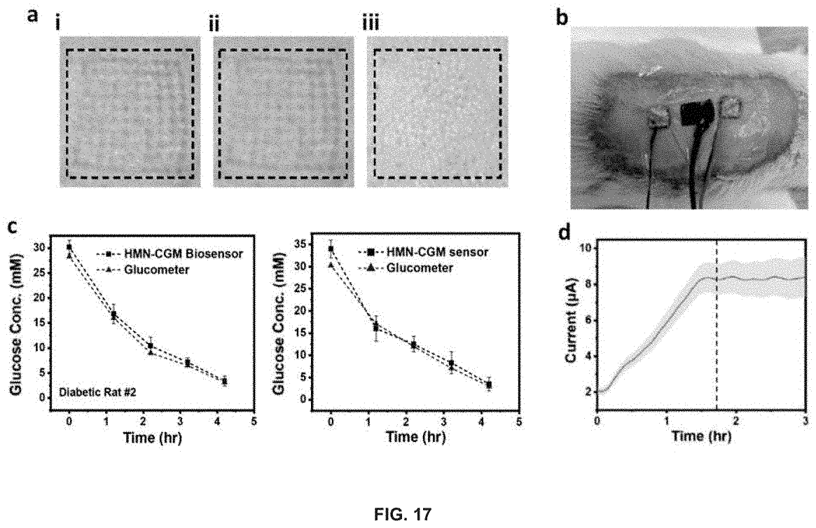

To study the skin penetration capability of the MNs, the three MN electrodes were inserted into the rat's dorsal skin, and it was observed that all the patches effectively penetrated through the skin as shown by the needle traces (FIG. 17a). Upon the patch removal, the needle traces were vanished within 10 min for all the MN electrodes, demonstrating the rapid skin recovery following patch application. The HMN-WE was a swellable MN array and in this type of MNs the needles tend to remain intact following transdermal application.

### Real-Time and Continuous Glucose Measurement in Diabetic Rats—Glucose Sensing

Having demonstrated the capability of the hydrogel microneedle—continuous glucose meter (HMN-CGM) for real-time and continuous measurement ex-vivo, the in-vivo performance of the device was studied. HMN-CGM assay was applied for real-time measurement of glucose using a rat model of diabetes. The diabetic rats were fasted for 5 hr prior to the experiment and treated with 4-5 IU kg⁻¹ dose of human recombinant insulin subcutaneously to lower blood glucose level from hyperglycemia to hypoglycemia range.

Before the experiment, the rats were sedated by injection of ketamine/xylazine. The HMN-CGM device was then applied into the rat's shaven dorsal skin (FIG. 17b) at different time points while the chronoamperometry measurement was performed for 40 s (FIG. 17c and FIG. 24). A calibration curve (FIG. 30g) was used to calculate the glucose concertation measured by HMN-CGM. In parallel, blood was collected from the rat tail for the blood glucose measurement using a handheld glucose meter. The results showed that the measurements extracted from HMN-CGM device matched the ones from glucose meter well. In the next experiment, the HMN-CGM device was applied for prolonged and continuous measurement of glucose in a healthy rat (FIG. 17d). It was observed that approximately after 100 min of device administration, the measured current reached its maximum level and stayed at this level for the next 80 min.

### Expanded Figure Descriptions

### FIG. 13: Electrical and Material Characterization of the HMN Polymer and Needles.

FIG. 13 depicts: a) EIS measurement of DA-HA PEDOT:PSS thin films using an Ag/AgCl RE and a Pt CE Data expressed as mean±SEM with n=3 replicates, the shadow line shows SEM. b) 4-point probe measurements of DA-HA PEDOT:PSS thin films. Data expressed as mean±SEM with n=3 replicates. c) Mechanical compression test for HMN patches, n=1 replicates. R2=0.955 as analyzed by simple linear regression. d) Swelling capability of HMN patches after a 10 min insertion into agarose hydrogel. Data is expressed as mean±SEM with n=3 replicates.

### FIG. 14: SEM and Optical Characterization of the HMN Polymer and Needles.

FIG. 14 depicts: a) SEM images showing the porosity of (i) 0%, (ii) 2.5%, and (iii) 5% PEDOT:PSS HMN polymer network. Scale bar of zoomed-in (top) and zoomed-out (bottom) is 100 μm and 40 μm, respectively. b) 2D optical microscope images of (i) 0%, (ii) 2.5%, and (iii) 5% PEDOT:PSS HMN (scale bar is 300 μm). c) 3D SEM images of (i) 0%, (ii) 2.5%, and (iii) 5% PEDOT:PSS HMN (scale bar is 200 μm). Insets show an enlarged view of a singular needle (scale bar is 50 μm).

### FIG. 15: In-Vivo pH Sensing Model using the Three MN Electrode System.

FIG. 15 depicts: a) HMN-WE trace in rat skin at (i) 0 min, (ii) 3 min, (iii) 5 min, and (iv) 10 min upon patch removal. b) H&E staining of rat skin showing the cavity created by the HMN penetration. Scale bar is 50 μm. c) Three MN electrode set-up on the dorsal side of the rat, connected to the potentiostat. d) Commercial pH meter probe set-up on the dorsal side of the rat, e) Validation of the measurements calculated from the HMN-pH meter's measured current by comparison to pH values measured by a commercial pH meter. Data expressed as the mean with n=3 replicates for pH using the commercial pH meter and n=2 replicates for current using the HMN-pH meter. f) Extended chronoamperometry for HMN in rats over 180 mins. Data expressed as mean±SEM with n=3 replicates.

FIG. 16. Characterizing the Formation of Pt NPs within the Composite Hydrogel.

FIG. 16 depicts: a) XPS measurement of DHP composite hydrogel in the absence of Ag (i) and in the presence of Ag b) SEM images of DHP composite hydrogel in the absence of Ag (i) and in the presence of Ag (ii), Scale bar: 10 μm. c) XRD measurement of samples with Ag and Pt ions (DHP Ag—Pt) shows diffraction peaks corresponding to the presence of metallic NPs. d) FTIR measurement of the composite hydrogel sample before and after addition of metal ions. e) EDX mapping analysis of DHP Ag-Pt composite hydrogel shows the formation of NPs (i) and presence of Pt (ii), Ag (iii), sulfur (iv) found in PEDOT:PSS structure, and nitrogen (v) presented in DA-HA hydrogel network. Scale bars: 10 μm (i, top) and 2 μm (i, bottom).

### FIG. 17. In-Vivo Glucose Detection in Diabetic Rats.

FIG. 17 depicts: a) HMN-WE patches were applied into the dorsal skin of rat for 10 min and then removed to observe the HMN trace. Magnified images of the trace of a patch into the skin after 0 min (i), 5 min (ii), and 10 min (iii). b) HMN-CGM assay with three MN electrodes was applied into the dorsal skin of rats and fixed with Tegaderm tapes to enable glucose measurement. c) HMN-CGM measurement of glucose levels in two different diabetic rats over 4.5 hr. The sedated rats were injected with 4-5 IU kg⁻¹ dose of human recombinant insulin (at t=5 min: after baseline measurement) subcutaneously to reduce the blood glucose level. For each time point, three different HMN-WEs were used to measure the glucose level. Error bar shows SD among three measurements. For each timepoint, blood samples were collected from the tail vein before and after applying HMN-CGM device, measured the glucose levels using a hand-held glucose meter, and reported the average to compare with HMN-CGM measurement. d) HMN-CGM assay was used for tracking of the level of glucose in a healthy rat over 3 hr. After 100 min, the signal reaches to a plateau. The blood glucose was measured 10.8±0.8 mM. The shadow shows SD among three measurements.

FIG. 25. In-vitro and ex-vivo characterization of pH sensing using the 5 wt % PEDOT:PSS HMN three-electrode system. FIG. 25 depicts: a) pH step-increase for HMN thin film after successive additions of 1M NaOH at 0.4 V, Ag/AgCl CE and Pt RE used, b) Extended swelling experiment for HMN on porcine skin over 120 mins. Data expressed as mean±SEM with n=3 replicates. c) Three MN electrode set-up from a (i) top view and (ii) side view on porcine skin, attached to the potentiostat. Ag/AgCl RE and Pt CE used, d) Extended chronoamperometry for HMN on porcine skin over 120 mins. Data expressed as mean±SEM with n=4 replicates, the shadow line shows SEM. a) Ex-vivo amperometric response of the three-electrode system at 0.4 V on pH equilibrated porcine skin. Inset shows corresponding pH calibration plot. Data expressed as mean±SEM with n=4 replicates. R₂=0.9311 as analyzed by non-linear regression.

FIG. 30. Ex-Vivo Glucose Detection using HMN-CGM Device.

FIG. 30 depicts a) SEM imaging was used to characterize the porosity of different composite hydrogel samples with different rates of DA conjugation. The porosity of 25% DA-HA (i), 17% DA-HA (ii), 10% DA-HA (iii), 17% DHP (iv), and 17% DHP Ag—Pt (v) samples were calculated 40%, 54%, 67%, 41%, and 21%, respectively. Scale bars: 50 μm b) DHP Ag—Pt HMN fabrication process. DA-HA was first mixed with 5% PEDOT: PSS solution. Second, Ag and Pt ion solutions were added, and the pH of solution was increased to 8 to make a chemically crosslinked network. The DHP Ag—Pt solution with adjusted pH was then added to PDMS mold and centrifugation was performed to remove air bubbles. Before drying a copper wire was added to make electrical connection. c) SEM images of HMN patches fabricated with DA-HA (i), DHP (ii), and DHP Ag—Pt (iii) solution demonstrating sharp needles suitable for skin penetration. Scale bars: 300 μm (top) and 150 μm (bottom). d) Short-term swelling capability of HMN arrays with different DA conjugation rates and DHP and DHP Ag—Pt HMNs were studied via applying the patches in agarose hydrogel for 10 min. Data is shown as the mean±SD, n=4. P-values were determined using one-way analysis of variance (ANOVA), *P<0.05, and **P<0.01. e) Mechanical compression test for HMN arrays with different DA conjugation rates and DHP and DHP Ag-Pt HMNs. f) HMN-CGM consisting of HMN-WE fabricated with DHP Ag-Pt composite hydrogel, an Ag/AgCl MN-RE, and an Au coated MN-CE was applied in porcine ear skin equilibrated with different glucose concentrations and chronoamperometry measurement was performed at 0.4 V for 40 s. g) The calibration curve correlating the current to glucose concentration (R²=0.96). This calibration curve was employed to estimate the glucose level extracted from in-vivo measurements. h) Swelling capability of HMN-WE was evaluated by applying the patch through porcine skin for different durations, i) HMN-CGM device was used for long-term glucose measurement in porcine skin equilibrated with different glucose concentrations. The inset shows that the slope of graphs in the first 10 min is proportional to the glucose level. Error bars and shadow in (i) show SD and each experiment was repeated at least three times.

### Example 3 References

[3] H. Chang, M. Zheng, X. Yu, A. Than, R. Z. Seeni, R. Kang, J. Tian, D. P. Khanh, L. Liu, P. Chen, C. Xu, Adv. Mater. 2017, 29, 1.

[20] M. Zheng, Z. Wang, H. Chang, L. Wang, S. W. T. Chew, D. C. S. Lio, M. Cui L. Liu, B. C. K. Tee, C. Xu, Advanced Healthcare Materials 2020, 9, 1901683.

[33] D. Mantione, I. Del Agua, A. Sanchez-Sanchez, D. Mecerreyes, Polymers 2017, 9, 354.

[6] H. Y. Son, J. H. Ryu, H. Lee, Y. S. Nam, ACS Appl. Mater, Interfaces 2013, 5, 6381.

[37] I. A, Sato, M. Werheid, C. Dosche, M. Oezaslan, Nanoscale Adv. 2019, 1, 3095.

[38] F. Merkoci, J. Patarroyo, L. Russo, J. Piella, A. Genc, J. Arbio N. G. Bastus, V. Puntes, Mater. Today Adv, 2020, 5, 100037.

[39] A. Ghavaminejad, C. H. Park, C. S. Kim, Biomacromolecules 2016, 17, 1213.

[40] G. Kaur, R. Adhikari, P. Cass, M. Bown, P. Gunatillake, RSC Advances 2015, 5, 37553.

[41] T. Nezakati, A. Seifalian, A. Tan, A. M. Seifalian, Chem. Rev2018,. 118, 6766.

[42] S. W. T. Chew, A. H. Shah, M. Zheng, H. Chang, C. Wiraja, T, W. J. Steele, C. Xu, Bioengineering & Translational Medicine 2020, 5, el 0157.

[43] J. Zaragoza, S. Fukuoka, M. Kraus, J. Thomin, P. Asuri, Nanomaterials 2018, 8, 1.

[44] S. P. Davis, B. J. Landis, Z. H. Adams, M. G. Allen, M. R. Prausnitz, J. Biomech, 2004, 37, 1155.

[45] N. Hermanns, L. Heinemann, G. Freckrnann, D. Waldenmaier, D. Ehrmann, J. Diabetes Sci. Technol. 2019, 13, 636.

[47] Y. Kim, M. R. Prausnitz, Nat Biomed Eng 2021, 5, 3.

[48] G. L. Long, J. D. Winefordner, Anal. Chem. 1983, 55, 712A.

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

What is claimed is:

1. A microneedle electrode comprising:
a hydrogel;
a probe coupled to the hydrogel, the probe for generating an electrochemical signal in the presence of the target; and
a conductive material for communicating the electrochemical signal through the hydrogel.

2. The microneedle electrode of claim 1, wherein the electrochemical signal is generated in-situ.

3. The microneedle electrode of claim 1, further comprising metal nanoparticles, wherein the metal nanoparticles optionally comprise platinum, silver, gold, palladium, or combinations thereof.

4. The microneedle electrode of claim 1, wherein the probe comprises an electroactive species, a redox active species, or a combination thereof.

5. The microneedle electrode of claim 1, wherein the probe comprises dopamine, conjugated dopamine, functionalized dopamine, crosslinked dopamine, metal-complexed dopamine, or combinations thereof.

6. The microneedle electrode of claim 1, wherein the hydrogel comprises an optionally functionalized polymer, wherein the polymer is gelatin, hyaluronic acid, alginate, chitosan, collagen, or combinations thereof.

7. The microneedle electrode of claim 6, wherein the hydrogel comprises methacrylated hyaluronic acid, or dopamine-functionalized hyaluronic acid.

8. The microneedle electrode of claim 1, wherein the probe coupled to the hydrogel comprises the probe being coupled to the hydrogel by a linker.

9. The microneedle electrode of claim 1, wherein the probe coupled to the hydrogel comprises the probe being coupled to metal nanoparticles.

10. The microneedle electrode of claim 1, wherein the electrode is for sensing pH, or the target.

11. The microneedle electrode of claim 10, wherein the target is a biomolecule present in interstitial fluid, wherein the biomolecule is optionally glucose, dopamine, uric acid, xanthine, hydrogen peroxide, potassium, or a combination thereof.

12. The microneedle electrode of claim 1, wherein the microneedle has a length of about 300 μm to about 1000 μm.

13. The microneedle electrode of claim 1, wherein the conductive material comprises a metal nanoparticle, graphene, a conductive polymer, or a combination thereof.

14. The microneedle electrode of claim 13, wherein the conductive polymer comprises poly(3,4-ethylenedioxythiophene) polystyrene sulfonate, polyacetylene, polypyrrole, polyindole, polyaniline or combinations or copolymers thereof.

15. A method of producing a microneedle, the method comprising:
combining a functionalized hydrogel and a conductive material to form a mixture;
adjusting the pH of the mixture; and
crosslinking the mixture in a mold to form a crosslinked material.

16. The method of claim 15, wherein crosslinking the mixture comprises exposing the mixture in the mold to UV light.

17. The method of claim 15, wherein combining the functionalized hydrogel and the conductive material comprises:
dissolving about 10-100 g/L of functionalized hydrogel in water or an aqueous solution with about 0.1 vol % to about 35 vol % conductive material; and
optionally, adding glycol in an amount of 0-5 vol %.

18. The method of claim 15, further comprising combining the functionalized hydrogel and the conductive material with a metal nanoparticle or metal nanoparticle precursor.

19. An apparatus for biosensing, the apparatus comprising:
the microneedle electrode according to claim 1;
a reference electrode;
a counter electrode; and
a detector for detecting the electrochemical signal.

20. A transdermal patch comprising the microneedle electrode according to claim 1.

21. The transdermal patch of claim 20, wherein the microneedle electrode is a working electrode, and the transdermal patch further comprises a reference electrode and a counter electrode, wherein the reference electrode optionally comprises Ag/AgCl. and the counter electrode optionally comprises Au.

22. A method for transdermal biosensing of a target in a subject, the method comprising:
applying the transdermal patch according to claim 20 to the skin of the subject:
detecting the electrochemical signal; and
associating the electrochemical signal to pH or to the concentration of the target in the subject.

23. The method of claim 22, wherein detecting the electrochemical signal is in-situ.
